(19) United States
(12) Patent Application Publication
(10) Pub. No.: US 2021/0204941 A1
(43) Pub. Date: Jul. 8, 2021

(54) CONTROLLABLE STEERABLE FUSING DEVICE

(71) Applicant: Steerable Instruments NV, Sint-Denijs-Westrem (BE)

(72) Inventors: Frank Dewaele, De Pinte (BE); Cyriel Mabilde, Oudenaarde (BE); Bart Blanckaert, Eeklo (BE); Lieven Maene, Knokke (BE); Alain Kalmar, Gent (BE)

(73) Assignee: Steerable Instruments NV, Sint-Denijs-Westrem (BE)

(21) Appl. No.: 17/059,911

(22) PCT Filed: May 29, 2019
(86) PCT No.: PCT/EP2019/064034

§ 371 Date: Nov. 30, 2020

(30) Foreign Application Priority Data
Jun. 1, 2018 (EP) 18175583.6

(51) Int. Cl. A61B 17/072; A61B 34/30; A61B 34/00; A61B 17/00

(52) U.S. Cl. CPC A61B 17/07207; A61B 34/30; A61B 34/71; A61B 2017/07257; A61B 2017/00327; A61B 2017/07285; A61B 2017/07271; A61B 17/00234

(57) ABSTRACT

Provided is a steerable fusing device (50) comprising: a steerable instrument (100) having a proximal end (20) and a distal (40) end comprising a shaft (130), a bendable proximal part (120) and a bendable distal part (140), the steerable instrument (100) configured such that the bendable distal part (140) bends responsive to bending of the bendable proximal part (120), fusing head (302) attached in fixed rotational relation to the bendable distal part (140) configured for fusing tissue captured between jaws (310, 320) of the fusing head (300), wherein the fusing head (300) is rotatable when the bendable distal part (140) is in a bent position by a complementary rotation of the bendable proximal part (120).

### FIELD OF THE INVENTION

The invention is in the field of controllable steerable fusing device (e.g. stapler, vessel sealer) for industrial, engineering and medical uses, more in particular for minimally invasive surgery. Most preferably, the controllable steerable stapler is robotically controllable, however, manual control is also envisaged.

### BACKGROUND TO THE INVENTION

There is an increased demand for systems that allow remote stapling in confined spaced having restricted access. The most common use is in the field of minimally invasive surgery, however, such systems also find applications in mechanical engineering to repair machines where disassembly is impractical or uneconomic for example in manufacturing installations, within building structures, submarine environments, in space, and the like. Such systems may be controlled robotically or manually. A steerable fusing device (e.g. stapler, vessel sealer) can be introduced through a small aperture, and the fusing device head deployed on one or more objects to be joined together. Problems in the art with existing steerable instruments especially those that are robotically controlled include complexity, expense, wear and tear, cleaning, sterilisation, movement reproducibility, tip stability and range of motions.

The da Vinci surgical system (Intuitive Surgical Inc, CA, USA) is currently a predominant robotic surgical system having a plurality of robotic arms each of which is attachable to a disposable laparoscopic instrument that is a steerable instrument. The prior art laparoscopic instrument comprises a shaft having a proximal and distal end, a proximal-end housing that is repeatably attachable to one of the robotic arms of the system. The proximal-end housing is disposed with 4 rotary dials to separately control rotation of an end effector (gripper), revolute movement of the shaft, and a separate rotation of the gripper arms to actuate them. A system of wires and pulleys within the disposable laparoscopic instrument transfers forces from the dials towards the end effector (gripper). The systems is highly complex; the proximal-end housing and effector end contain a large number of components held under tension by the wires to transmit rotational forces through a rotatable shaft and across revolute joints to the distal wrist and end effector which also contains a complex arrangement. A known disadvantage is the high cost of the instrument because of the multitude of components that have high tolerances and reliability and are able to withstand forces during use and harsh chemical and temperature cleaning protocols, as well as the assembly time. Hence it is an aim to provide a lower cost stapler for robotic use, and also for use manually.

Because the laparoscopic instrument of the art is complex and expensive it is operationally cost-effective (i.e. a necessity) to reuse it. The disposable laparoscopic instrument contains a large number of moving components and revolute joints under tension, each subject to wear and tear. A movement of the instrument must be reproducible; a returning movement must bring the instrument to the same place and in the same orientation. The mechanical joints in the proximal-end housing and distal wrist/end effector are each a source of error and contribute to a mechanical play or backlash which reduces positional accuracy and reproducibility. As such reusability is limited to a fixed number times. Moreover, a use-counter must be implemented to limit the number of times of reuse.

With reusability is a problem of cleaning and sterilisation. Means must be provided to allow the instrument to be thoroughly cleaned, sterilised and dried, including the innards of the proximal-end housing. The cleaning requires supplementary strategies compared with standard instruments consuming a high number of manual hours; cleaning solution is pumped into the proximal-end housing and further thoroughly rinsed away. It is known in the literature that some cleaning processes are not effective; blood clots have been observed on an inner shaft of a cleaned instrument. The cleaning solution is corrosive, reducing the lifespan of the components. Limited access to the innards impedes the rinsing and drying process; there is a risk that residue of cleaning solution remains.

For some procedures, a continuous rotation of the shaft or of the end effector is desirable, for instance, for passage through a tortuous cavity. The prior art pulley and wire system has a limit on the number of revolution of the shaft currently to 1.5 rotations. An infinitely rotatable shaft and end effector would be desirable for many applications.

There is a need in the art for a manually or robot controllable steerable stapler having a simpler mechanical coupling yet offers control of the instrument direction and of the stapler head direction, that is cheaper to manufacture, has an infinitely rotatable stapler head, is safer and overcomes the problems of the art.

### SUMMARY

Provided herein is a steerable fusing device (50) comprising:
a steerable instrument (100) having a proximal end (20) and a distal (40) end comprising a shaft (130), a bendable proximal part (120) and a bendable distal part (140), the steerable instrument (100) configured such that the bendable distal part (140) bends responsive to bending of the bendable proximal part (120), and
a fusing head (302) attached in fixed rotational relation to the bendable distal part (140) configured for fusing tissue captured between jaws (310, 320) of the fusing head (300),

wherein the fusing head (300) is rotatable when the bendable distal part (140) is in a bent position by a complementary rotation of the bendable proximal part (120).

Further provided here is a steerable fusing device (50) comprising:
a steerable instrument (100) having a proximal end (20) and a distal (40) end comprising a shaft (130), a bendable proximal part (120) configured to bend omnidirectionally in a curve and a bendable distal part (140) configured to bend omnidirectionally in a curve, the steerable instrument (100) configured such that the bendable distal part (140) bends responsive to bending of the bendable proximal part (120), and
a fusing head (302) attached in fixed rotational relation to the bendable distal part (140) configured for fusing tissue captured between jaws (310, 320) of the fusing head (300),

wherein the fusing head (300) is axially rotatable when the bendable distal part (140) is in a bent position by a complementary axial rotation of the bendable proximal part (120).

The steerable instrument (100) may be further configured such that the direction of the fusing head (300) is changeable while the shaft is in a fixed rotational position by a complementary movement of the connector (110).

The steerable fusing device (50) may further comprise a connector (110) configured for dismountable attachment to a robotic arm, attached in fixed rotational relation to the bendable proximal part (120), wherein
the bendable distal part (140) bends responsive to bending of the bendable proximal part (120), and the stapler head (300) is (axially) rotatable when the bendable distal part (140) is in a bent position by a complementary (axial) rotation of the connector (110),
the shaft (130) is pivotable around a fulcrum zone (134) on the shaft (130) and changes direction responsive to a complementary movement of the connector (110), thereby providing control of the shaft (130) direction, bending of the bendable distal part (140), and rotation of the end effector (150) through robotic movement of the connector (110).

The connector (110) may comprise a rigid member for dismountable non-rotational attachment to a complementary fitting on the robotic arm, wherein the complementary fitting is disposed in fixed relation to a last joint of the robotic arm.

The steerable fusing device (50) may further be provided with a drive shaft (460) attached at its distal end (40) to the stapler head (50) for transmission of force to control jaws (310, 320) of the stapler head (50).

The BDP (130) may comprise a plurality tandemly arranged joints (400′, a-f), it may be configured to be bendable in a curve, and joints (400′, a-f) may form an essential continuous lumen (428) for passage of the drive shaft (460).

Each joint (400′, a-f) may be formed from two articulating joint parts (400, a-f), each articulating joint parts (400, a-f) is provided with a separate lumen (422a, 422b) for passage of the drive shaft (460) wherein the proximal end (20), distal end (40) or both ends of the lumen (422a, 422b) wall flares outwards.

A maximum bending angle of a joint (400′, a-f) may be limited to 30°.

A transverse profile of the drive shaft (460) may be 25% to 99.8% of a transverse profile of the lumen (420) of the joint.

The fusing head (300) may be a stapler head (302) comprising a staple cartridge jaw (320) configured to support a staple cartridge holding a plurality of surgical staples (334) and an anvil jaw (310) disposed with an anvil plate, the anvil jaw (310) or staple cartridge jaw (320) being moveable with respect to the other jaw between an open and closed position.

The staple cartridge jaw (320) or staple cartridge may comprise a rotatable threaded support (322) and a sled member (324) whereby rotation of the rotatable threaded support (322) advances the sled member (324) to deploy staples (334) from the staple cartridge (320) and into the anvil plate.

The steerable fusing device (50) may further comprising a slidable constraining member (350) configured to move an open end (34) of the anvil jaw (310) or staple cartridge jaw (320) closer to the other jaw.

The slidable constraining member (350) may be disposed in co-operation with the rotatable threaded support (322) such that rotation of the rotatable threaded support (322) advances the slidable constraining member (350) to move an open end (34) of the anvil jaw (310) or staple cartridge jaw (320) closer to the other jaw.

The slidable constraining member (350) may comprise a spacing beam (352) flanked by a pair of stop members (354, 356), where the spacing beam (252) is disposed in a slot (316, 326) of a body (318, 328) of each of the anvil jaw (310) and staple cartridge jaw (320) and each stop member (354,356) abuts the slot edges (316a,b, 326a,b) thereby retaining the anvil jaw (310) and staple cartridge jaw (320) together at a distance determined by spacing beam (352).

The staple cartridge jaw (320) and anvil jaw (310) may be connected at a jointed end (32) to one or more joints (340) allowing movement of the jaws (310, 320) between an open and closed position, wherein the jointed end (32) is disposed proximal (20) or distal (40) to an opening end (34) of the jaws.

Further provided is a steerable fusing device (50) comprising:
a steerable instrument (100) having a proximal end (20) and a distal (40) end comprising a shaft (130), a bendable distal part (140) configured to bend omnidirectionally in a curve, a set of longitudinal members, LMs, configured to transmit actuating movement along the shaft (130) to the bendable distal part (140), the steerable instrument (100) configured such that the bendable distal part (140) bends responsive to actuation of the proximal part (120), and
a fusing head (302) attached in fixed rotational relation to the bendable distal part (140) configured for fusing tissue captured between jaws (310, 320) of the fusing head (300). A proximal end of the set of LMs may be configured for detachable coupling to a set of actuators, wherein each actuator in the set of actuators controls movement of one or more LMs, thereby controlling bending of the bendable distal part (140). At least one actuator in the set of actuators may be a servo motor, linear actuator, hydraulic actuator, or pneumatic actuator. The set of actuators may be incorporated into a complementary fitting of a robotic arm. The steerable fusing device (50) may incorporate the features described herein. Further provided is a system comprising a robotic arm and a steerable fusing device (50) as defined herein.

### FIGURE LEGENDS

FIG. 1 is an illustration of a steerable fusing device (50) that is a steerable stapler (52) as described herein comprising a steerable instrument (100) and fusing head (300) that is a stapler head (302).

FIG. 2A is a further illustration of a steerable stapler (52) of FIG. 1 disposed with a connector configured for dismountable attachment to a robotic arm.

FIG. 2B shows the steerable stapler (52) of FIG. 2A wherein the BDP (140) is bent responsive to a bending of the BPP (120).

FIG. 3 illustrates a fusing head (300) that is a stapler head (302) as described herein where the jointed end (32) is oriented towards the proximal end (20) and the opening end (34) is oriented towards the distal end (40).

FIG. 4 illustrates a stapler head (302) as described herein where the jointed end (32) is oriented towards the oriented towards the distal end (40) and the opening end (34) is oriented towards the proximal end (30).

FIGS. 5A and 5B depict a stapler head (302) as described herein in the closed configuration; in FIG. 5A the sled member (324) is partially advanced in towards the opening end and in FIG. 5B the sled member (324) is fully advanced in the opening end (34) direction.

FIG. 6 depicts a stapler head (302) as described herein where the staple cartridge jaw (320) anvil jaw (310) are each disposed with a tissue clamping member (321).

FIG. 7 depicts a slidable constraining member (350) that is a serif i-beam.

FIGS. 8A and 8B are upper and lower plan views of the stapler head (302) showing constraining member (350).

FIGS. 9A and 9B show advancement of the constraining member (350) to move of the staple cartridge jaw (320) against the anvil jaw (310)

FIGS. 9C and 9D, show advancement of the constraining member (350) to the anvil jaw (310) against the staple cartridge jaw (320).

FIGS. 10A,A′ and 10B,B′ each depict a stapler head (302) disposed with the constraining member (350) that is a serif i-beam and a slot (316) is disposed with a position discrete recess (317). Advancement of the serif i-beam initially closes the jaws (FIGS. 10A,A′), and further advancement opens the jaws (FIGS. 106,6′) to release the stapled object.

FIGS. 11A, A′ and 11B, B′ each depict a stapler head (302) disposed with the constraining collar (370). In FIGS. 11A and A′ the staple cartridge jaw (320) moves to close the jaws, whereas in FIGS. 11B and B′ anvil jaw (310) moves to close the jaws.

FIGS. 12A, A′ and 12B, B′ each depict a stapler head (300) disposed with the closing screw (380). In FIGS. 12A and 12A′ the staple cartridge jaw (320) moves to close the jaws, whereas in FIGS. 12B and 12B′ anvil jaw (310) moves to close the jaws.

FIG. 13 shows the BDP (140) in a bent position and comprising a plurality of tandemly arranged joints (400,a-f).

FIG. 14 is a side view of a part of a joint (400) that is an articulated LM guide (405).

FIG. 15 is a plan view of a part of a joint (400) that is an LM guide (402) rectangular-profiled channels.

FIG. 16 is a plan view of a part of a joint (400) that is an LM guide (402) having circular-profiled channels.

FIG. 17 is a cross-sectional view of a plurality of joint parts (400a,b) each part being an LM guide (402a,b).

FIG. 17A is an enlarged view of the continuous lumen (428) of FIG. 17.

FIGS. 18A and B each show the lumen of each articulated joint part (LM guide (405)) is chamfered at both proximal and distal ends; in FIG. 18A the bendable part is straight, and in FIG. 18B the bendable part is bent.

FIGS. 19A and B shows each the lumen of each articulated joint part (LM guide (405)) is uniformly cylindrical. In FIG. 19A the bendable part is straight, and in FIG. 19B the bendable part is bent and locks the drive shaft.

FIG. 20 is a cross-sectional view of a steerable fusing device that is a steerable stapler.

FIGS. 19A and B shows each the lumen of each articulated joint part (400) (articulated LM guide (405)) is uniformly cylindrical (FIG. 19A). Bending (FIG. 19B) causes the wall of the lumens to bite or lock (429) against the drive shaft (460) thereby restricting its movements.

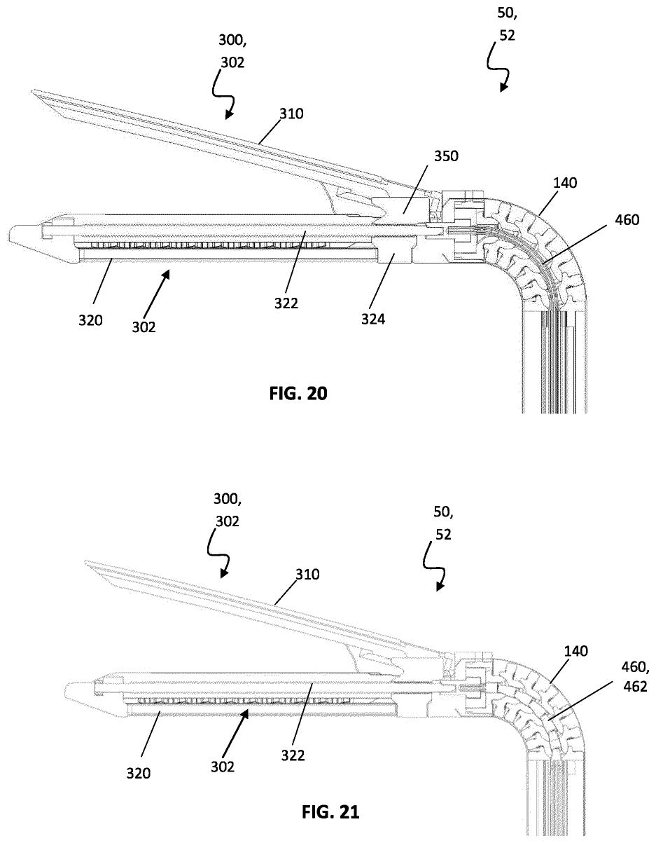

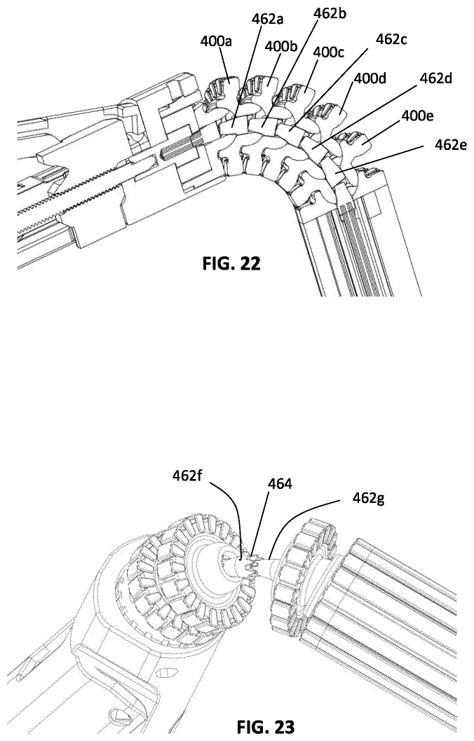

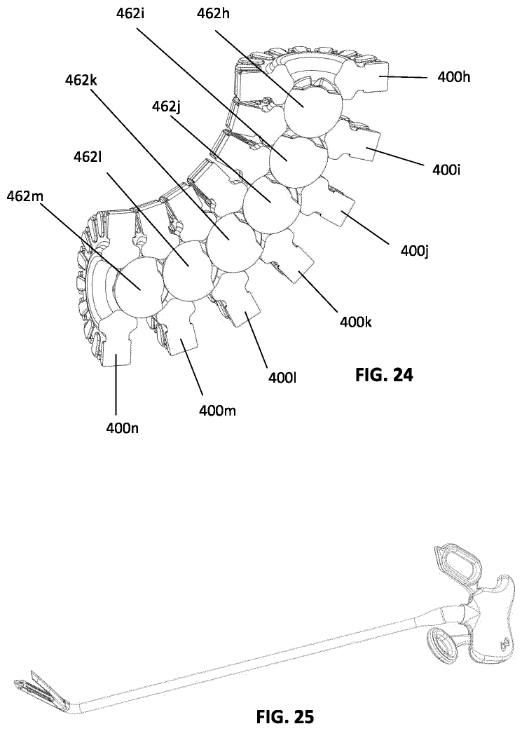

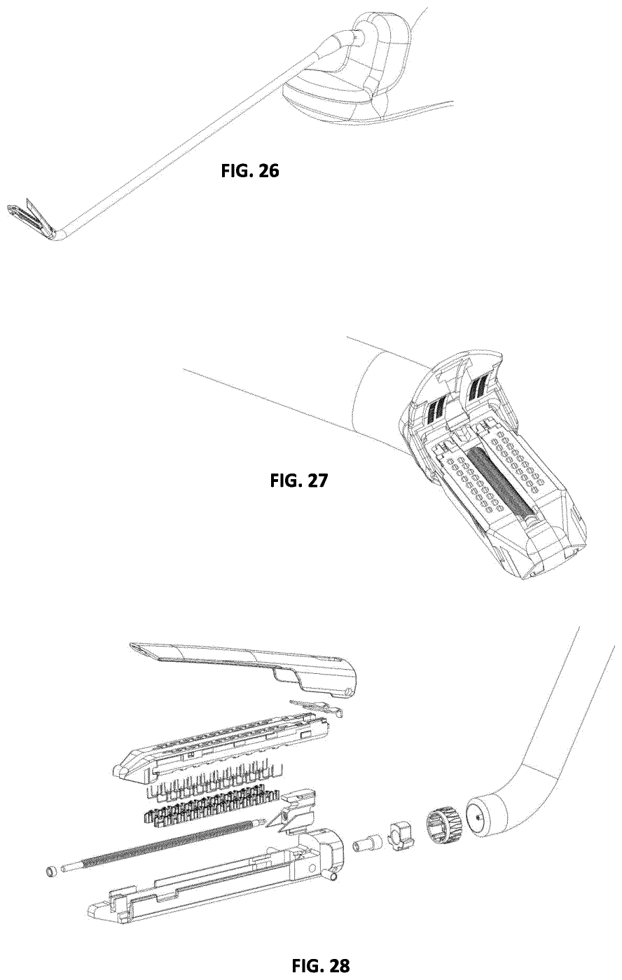

FIG. 20 is a cross sectional view of a steerable fusing device provided with a cable-like drive shaft.

FIG. 21 is a cross sectional view of a steerable fusing device provided with a segmented drive shaft.

FIG. 22 is a detail of FIG. 21 showing separate articulated segments of the drive shaft.

FIG. 23 is a detail of a gear-type universal joint between two articulated segments of the drive shaft.

FIG. 24 is a cross sectional of a segmented drive shaft showing separate cylindrical articulated segments .

FIG. 25 is an isometric view of a steerable fusing device that is a steerable stapler dismountably attached at the proximal end to a handle.

FIG. 26 is an isometric view of a steerable fusing device attached to a robotic arm.

FIG. 27 is an isometric view of a stapler head.

FIG. 28 is an isometric view of exploded parts of a stapler head.

### DETAILED DESCRIPTION

Before the present system and method of the invention are described, it is to be understood that this invention is not limited to particular systems and methods or combinations described, since such systems and methods and combinations may, of course, vary. It is also to be understood that the terminology used herein is not intended to be limiting, since the scope of the present invention will be limited only by the appended claims.

As used herein, the singular forms “a”, “an”, and “the” include both singular and plural referents unless the context clearly dictates otherwise.

The terms “comprising”, “comprises” and “comprised of” as used herein are synonymous with “including”, “includes” or “containing”, “contains”, and are inclusive or open-ended and do not exclude additional, non-recited members, elements or method steps. It will be appreciated that the terms “comprising”, “comprises” and “comprised of” as used herein comprise the terms “consisting of”, “consists” and “consists of”.

The recitation of numerical ranges by endpoints includes all numbers and fractions subsumed within the respective ranges, as well as the recited endpoints.

The term “about” or “approximately” as used herein when referring to a measurable value such as a parameter, an amount, a temporal duration, and the like, is meant to encompass variations of +/−10% or less, preferably +/−5% or less, more preferably +/−1% or less, and still more preferably +1-0.1% or less of and from the specified value, insofar such variations are appropriate to perform in the disclosed invention. It is to be understood that the value to which the modifier “about” or “approximately” refers is itself also specifically, and preferably, disclosed.

Whereas the terms “one or more” or “at least one”, such as one or more or at least one member(s) of a group of members, is clear per se, by means of further exemplification, the term encompasses inter alia a reference to any one of said members, or to any two or more of said members, such as, e.g., any ≥3, ≥4, ≥5, ≥6 or ≥7 etc. of said members, and up to all said members.

All references cited in the present specification are hereby incorporated by reference in their entirety. In particular, the teachings of all references herein specifically referred to are incorporated by reference.

Unless otherwise defined, all terms used in disclosing the invention, including technical and scientific terms, have the meaning as commonly understood by one of ordinary skill in the art to which this invention belongs. By means of further guidance, term definitions are included to better appreciate the teaching of the present invention.

In the following passages, different aspects of the invention are defined in more detail. Each aspect so defined may be combined with any other aspect or aspects unless clearly indicated to the contrary. In particular, any feature indicated as being preferred or advantageous may be combined with any other feature or features indicated as being preferred or advantageous.

Reference throughout this specification to “one embodiment” or “an embodiment” means that a particular feature, structure or characteristic described in connection with the embodiment is included in at least one embodiment of the present invention. Thus, appearances of the phrases “in one embodiment” or “in an embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention, and form different embodiments, as would be understood by those in the art. For example, in the appended claims, any of the claimed embodiments can be used in any combination.

In the present description of the invention, reference is made to the accompanying drawings that form a part hereof, and in which are shown by way of illustration only of specific embodiments in which the invention may be practiced. Parenthesized or emboldened reference numerals affixed to respective elements merely exemplify the elements by way of example, with which it is not intended to limit the respective elements. It is to be understood that other embodiments may be utilised and structural or logical changes may be made without departing from the scope of the present invention. The following detailed description, therefore, is not to be taken in a limiting sense, and the scope of the present invention is defined by the appended claims.

The terms “distal” or “distal to” and “proximal” or “proximal to” are used throughout the specification, and are terms generally understood in the field to mean towards (proximal) or away (distal) from the operator's side of an apparatus. Thus, “proximal” or “proximal to” means towards the operator's side and, therefore, away from the workpiece or patient's side. Conversely, “distal” or “distal to” means towards the workpiece or patient's side and, therefore, away from the operator's side. The steerable stapler has a distal and proximal end and components of the stapler including the steerable instrument, stapler head have distal and proximal ends that correspond with distal and proximal ends of the steerable stapler.

Provided herein is a steerable fusing device controllable by a handle or robotic arm. The steerable fusing device comprises a steerable instrument that has a proximal end and distal end, disposed with a fusing head at the distal end. The steerable instrument comprises a shaft, a bendable proximal part (BPP) and a bendable distal part (BDP). The steerable instrument may comprise a connector attached to the BPP configured for dismountable attachment to the robotic arm or to a handle, or a handle may be permanently attached to the BPP; the handle is configured for manual control of the steerable instrument and fusing head. The steerable instrument may comprises a shaft and a bendable distal part (BDP). The BPP may be absent and movements of the BDP may be controlled by one or more actuators (e.g. linear actuators, servo motors) connected to longitudinal members (LMs) at the proximal end of the steerable instrument. The one or more actuators may be located in a complementary fitting on the robotic arm. The steerable fusing head is attached to the bendable distal part. The steerable instrument configured such that the bendable distal part bends responsive to bending of the bendable proximal part by the connector or handle, and the distal tip or fusing head is rotatable when the bendable distal part is in a bent position by a complementary rotation of the connector or handle. The rotation in the bent position is around a longitudinal (axial) axis of rotation e.g. an axis of rotation (112) of the BPP (120) and around an axis of rotation (152) of the BDP (140). An axial rotation in the bent position of the BPP (120) may induce a complementary axial rotation in the bent position of the BDP (140). It is further configured such that shaft pivots around a fulcrum zone responsive to movements of the connector or handle. Control of the shaft direction, displacement of the shaft along an axial (A-A′) direction, bending of the bendable distal part, and rotation of the distal tip or end effector is realised through movement of the single connector or handle.

The steerable fusing device may be reusable, buts is preferably single use. A single use steerable fusing device might be preferable if a surgeons wishes to use a fusing head with different lengths or staple heights.

The direction of the shaft refers to its angular placement. Changing a direction of the shaft is achieved typically by a pivoted rotation around a fulcrum zone. The fulcrum zone coincides with a longitudinal axis (A-A′) of the shaft, for instance, a central longitudinal axis of the shaft. Such movements have two degrees of freedom (2-DOF), and may be known as pitch and yaw. When referring to direction, two degrees of freedom is equivalent to a rotation about two axes. The fulcrum zone is where axes of rotation intersect. The fulcrum zone typically coincides with an entry point to the space being investigated, for instance with a hole made in a wall, membrane or port. The fulcrum is provided by the entry point. Where the steerable instrument is a laparoscopic medical instrument, the fulcrum zone is placed at a bodily incision or natural orifice (e.g. urthera) where the laparoscopic medical instrument is introduced. The minimally invasive instrument is typically enters the body via a trocar—a tube-like port inserted into an incision or natural orifice—that supports the steerable instrument and is amendable to pivoted rotation around the fulcrum point of the incision.

The axial position of the shaft refers to its axial (FIG. 2A, A-A′) positional placement. Changing an axial position of the shaft is achieved typically by displacing the shaft axially in a A-A′ direction. Such movement has one degree of freedom (1-DOF), and may be known as axial displacement. The entry point to the working space e.g. a bore hole, maintenance port, or a bodily incision supports the steerable instrument and allows the instrument shaft to slide relative to the entry point. Where the steerable instrument is a minimally invasive medical instrument, the medical instrument is introduced via a bodily incision or natural orifice. The medical instrument is typically enters the body via a trocar—a tube-like port inserted into an incision—that supports the steerable instrument and allows the instrument shaft to slide relative to the trocar.

The direction of the distal tip or fusing device head refers to its angular placement relative to the shaft. Changing a direction of the distal tip or fusing devicehead is achieved primarily by actuation of the BPP that changes the direction of BDP. A central axis (FIG. 2B, 152) of the distal tip or fusing devicehead in different directions intersect at a BDP zone of motion, ZOM, (142) that is a zone coinciding with a central axis (A-A′) of the shaft. Bending movements of the BDP has two effective degrees of freedom (2-DOF) around its zone of motion, and may be known as effective pitch and effective yaw of the distal tip or end effector that is different from the pitch and yaw of the instrument shaft. The inventors have found that a geometric centre of the BDP zone of motion (BDP-CZOM) can be used as an effective fulcrum point to robotically control the direction of the distal tip or end effector, even when the BDP bends along a curve. Advantageously, treating the direction of the distal tip or fusing devicehead as pivoting around BDP-CZOM allows the axes of rotation of the last 2 or 3 revolute joints of the robotic arm to intersect at the BPP-CZOM, thereby reducing the volume in which the links towards the robotic base move and hence reducing a risk of collision with objects including adjacent equipment and additional robotic arms.

The direction of the connector or handle refers to its angular placement relative to the shaft. Changing a direction of the connector is achieved primarily by the robotic arm. Changing a direction of the handle is achieved manually. A central axis (FIG. 2B, 112) of the connector (110) or handle in different directions intersect at a BPP zone of motion (122) that is a zone coinciding with a central axis (A-A′) of the shaft. Bending movements of the BPP has two effective degrees of freedom (2-DOF) around its zone of motion, and may be known as effective pitch and effective yaw of the connector or handle that is different from the pitch and yaw of the instrument shaft. The inventors have found that a geometric centre of the BPP zone of motion (BPP-CZOM) can be used as an effective fulcrum point to control the direction of the direction of the connector or handle, even when the BPP bends along a curve. Advantageously, treating bending of the connector or handle as pivoting around BPP-CZOM allows the axes of rotation of the last 2 or 3 revolute joints of the robotic arm to intersect at the BPP-CZOM, thereby reducing the volume in which the robotic links towards the base move and hence reducing a risk of collision with objects including adjacent equipment and additional robotic arms.

The BPP where present is disposed at a proximal end of the shaft. It is axially rotationally fixed to the proximal end of the shaft. An axial rotation of the shaft may cause an axial rotation of the BPP. The BPP may contact the shaft. The BPP may be adjacent to the shaft. Movement of the BPP induces a movement response in the BDP. Movement of BPP in different radial directions and to different bending degrees results in a corresponding change in radial direction and/or degree of bending of the BDP. The BPP may be cylindrical. The BPP may be configured to bend in a curve. The BPP may comprise a plurality tandemly arranged joints and is configured to bend in a curve. The BPP may be configured to bend around one or more tandemly arranged joints (e.g. ball and socket joints) each having 2DOF. The BPP may be configured to bend around two or more tandemly arranged joints (e.g. revolute joints offset by 90 deg) each having 1DOF. The BPP may be configured to bend along a moulded flexible member as disclosed, for instance, in US 2006/0095074. The BPP may be configured to bend along a curve.

The BPP may be absent, in which case the steerable instrument may comprises a shaft and a bendable distal part (BDP). Bending movements of the BDP may be controlled by one or more actuators (e.g. linear actuators, servo motors, hydraulic, pneumatic) connected or connectable at the proximal end of the steerable instrument to longitudinal members (LMs) (see later below). For instance, an actuator may be connected or connectable to one, two, three, four or more LMs at the proximal end of each LM. Typically the LMs in the shaft would be actuated i.e. moved by the actuator thereby causing the BDP to bend. In such case, the tool may be devoid of a BPP. Such an arrangement would allow integration into a surgical robot. The proximal end of the set of LMs is configured for detachable coupling to a set of actuators, wherein each actuator in the set of actuators controls movement of one or more LMs, thereby controlling bending of the bendable distal part (140). The set of actuators may be incorporated into a complementary fitting of a robotic arm.

The BDP is disposed at a distal end of the shaft. It is axially rotationally fixed to the distal end of the shaft. An axial rotation of the shaft may cause an axial rotation of the BDP. The BDP may contact the shaft. The BDP is adjacent to the shaft. The BDP moves in response to movement of the BPP. Movement of BPP in different radial directions and to different bending degrees results in a corresponding change in radial direction and/or degree of bending of the BDP. The BDP may be cylindrical. The BDP may be configured to bend in a curve. The shaft may be rigid. The BDP may comprise a plurality tandemly arranged joints and is configured to bend in a curve. The BDP may be configured to bend around one or more tandemly arranged joints (e.g. ball and socket joints) each having 2DOF. The BDP may be configured to bend around two or more tandemly arranged joints (e.g. revolute joints offset by 90 deg) each having 1DOF. The BDP may be configured to bend along a curve.

The steerable instrument may contain a motion amplifier region having a plane section larger than that of the BDP. In the motion amplifier region, consecutive plane sections gradually increase in size in the distal to the proximal direction. The motion amplifier region may be located within the shaft, or at least partially within the BPP. With the amplifier, movement of the connection and hence of the BPP results in a correspondingly larger movement of the BDP. Bending degree of the bendable distal part responsive to bending degree of the bendable proximal part is amplified by the motion amplifier region. An example of a motion amplifier region is set out in WO 2016/091858 A1 A which is incorporated herein by reference. Advantageously, the presence of a motion amplifier region reduces the movement volume of the robotic arm and hence reduces a risk of collision with objects including adjacent equipment and additional robotic arms.

The shaft may be rigid or semi-rigid, or may be flexible and become rigid or semi-rigid when co-operating with a rigid or semi-rigid exotube or outer tube, endotube or inner tube. The distal end of the shaft is disposed with the BDP. The proximal end of the shaft is disposed with the BPP. The shaft part is longitudinal, meaning it is longer in one direction. It does necessarily not imply the shaft part is straight. The shaft part may be straight or curved, for instance, having a C- or S-shape. The shaft may be straight. The shaft preferably has a circular transverse (perpendicular to a central axis) profile. The shaft may be cylindrical.

The steerable instrument is configured for (axial) rotation of the distal tip of the BDP or the fusing devicehead about its own (axial) axis when the BDP is in a bent position, by a complementary (axial) rotation of the BPP. It is appreciated that the distal tip of the BDP refers in this context to the distal terminal end of the BDP.

The fusing head may be rotationally fixed in relation to the BDP, and the fusing head is (axially) rotatable when the BDP is in a bent position, by a complementary (axial) rotation of the BPP. The end effector may be directly attached to the distal end of the BDP (without a coupling).

The fusing head may be dismountable, in which case the BDP is provided with a coupling for attachment to the fusing head. The coupling may be rotationally fixed in relation to the BDP, and the coupling is (axially) rotatable when the BDP is in a bent position, by a complementary (axial) rotation of the BPP. The fusing head to the coupling is rotationally fixed in relation to the BDP.

Rotationally fixing the coupling or fusing head relative to the BDP may be achieved using a permanent (non-adjustable) connection or joint, or by means of a lockable element configured to allow rotational adjustment of and to rotationally fix the coupling or end effector in rotational relation to the BDP.

The steerable instrument may further comprise a connector configured for dismountable attachment to the robotic arm, more in particular to a fitting on the robotic arm or to a detachable handle. The connector is rotationally fixed in relation to the BPP. The connector is rotationally fixed in relation to the proximal terminal end or tip of BPP. The connector is attached fixed in relation to the proximal terminal end or tip of BPP. The connector may be provided attached to the proximal terminal end or tip of BPP. The connector may be provided attached to the aforementioned cylindrical portion. Rotationally fixing the connector relative to the BPP proximal terminal end or tip may be achieved using a permanent (non-adjustable) connection or joint, or by means of a lockable element configured to allow rotational adjustment of and to rotationally fix the connector in rotational relation to the BPP.

The connector may comprise a rigid member. The rigid member is configured for dismountable attachment to a complementary fitting on the robotic arm or detachable handle. The rigid member is configured for non-rotational dismountable attachment to a complementary fitting on the robotic arm or detachable handle. The rigid member is configured for displaceable dismountable attachment to a complementary fitting on the robotic arm or detachable handle. The rigid member is configured for non-rotational dismountable attachment to a complementary fitting on the robotic arm or detachable handle. The connector that is a rigid member may have a straight cylindrical form, as shown, for instance, in FIG. 2A and 2B. The diameter of the cylinder may be the same as, grater than or smaller than the diameter of the proximal tip of the BPP. The connector that is a rigid member may have another shape, such as L- shape, C-shape, F-shape.

It is appreciable that the attachment to the robotic arm is to the effector end of the robotic arm, typically in connection—in a straight line or at an angle—with the end joint.

The same connector may be used to attach the steerable fusing device to a fitting on a robotic arm or to a handle.

Wherein the BPP is absent and the LMs are moved by a set of actuators, shaft may be attachable to the robotic arm.

The steerable instrument may further comprise a handle configured for manual control of the instrument by the operator. The handle is preferably rotationally fixed in relation to the BPP. The handle is preferably rotationally fixed in relation to the proximal terminal end or tip of BPP. The handle is preferably attached fixed in relation to the proximal terminal end or tip of BPP. The handle may be provided attached to the proximal terminal end or tip of BPP. The handle may be provided attached to the aforementioned cylindrical portion. Rotationally fixing the handle relative to the BPP proximal terminal end or tip may be achieved using a permanent (non-adjustable) connection or joint, or by means of a lockable element configured to allow rotational adjustment of and to rotationally fix the handle in rotational relation to the BPP.

The handle may be non-dismountably or dismountably attached to BPP.

The handle may contain a manual actuator such as a lever for closing and opening the jaws of the fusing head. The same manual actuator or a further manual actuator may be provided for deployment of the staples.

The handle may be disposed with a motor for closing and opening the jaws of the fusing head. The same motor or a further motor may be provided for deployment of the staples. The motor may be non-dismountable or dismountable from the handle.

The handle may be provided with a power source (e.g. non-rechargable or rechargeable battery). The battery may be non-dismountable or dismountable from the handle.

The fusing head is a device that joins or seals objects together. Examples of different types of fusing heads include a stapler head, linear stapler head, a circular stapler head, stapler head in which the stapling direction is perpendicular or oblique to the direction of the distal tip of the BDP, sealer head (e.g. for sealing a vessel using bipolar electricity, electrical welding or piezo welding). The fusing head typically comprises a set of jaws moveable between an open and closed position, configured to receive one or more objects for sealing in the open configured. Application of force brings the jaws to a closed position. In the closed position, the one or more objects are joined or sealed.

The fusing head typically has a jointed end and an opening end. The jaws are connected at the jointed end to one or more joints allowing movement of the jaws between the open and closed position. The jointed end may be disposed distal to an opening end of the jaws (see, for instance, FIG. 4); this configuration allows for better visualtion of the object to be fused as it is not obscured by the joint. Moreover, in combination with the bending capability, it allows the object to be scooped into the jaws, thereby requiring less space around the object. The jointed end may disposed proximal to an opening end of the jaws. The joint may be allow parallel opening of the jaws. The joint may be allow rotation of one or both jaws respect to each other to achieved opening.

Control of the opening and closing of the jaws of the fusing head is by a jaw-actuating mechanism disposed at the distal end of the steerable instrument, preferably in the fusing head. Examples of jaw-actuating mechanisms are listed below, some of which are further elaborated elsewhere herein:
A constraining member (e.g. I-beam) slidable in relation to one or both jaws that bring the jaws from an open to closed position by containing them together as the constraining member advances towards an opening end of the jaws. A cam provided on one or both jaws allows for gradual closing. The constraining member may be actuated by a rotatable threaded support (lead screw), electric motor, piezoelectro motor, drive shaft, a block and tackle mechanism.
A rotatable threaded support (lead screw) that moves T-bar disposed in a threaded carriage engaged with the rotatable threaded support, wherein the arms of the T-bar are engages in a hinged oblique slot or over a cam. Movement of the T-bar raises or lower the slot relative to the hinge. This provides a slow but strong clamping force.
By retracting the jaws in a constraining collar.
Providing an arrangement of gears. The gears output act on the joined end of the jaws. The gears can be selected to reduce the speed but increase the force. The gears can be driven by a drive shaft or an electric motor or piezoelectric motor.
Direct pulling. This allows for quick response but low force. The mechanisms can be similar to known mechanisms for actuating e.g. scissors. The pulling cable pulls a T bar through an oblique slot. Or it pulls on a lever. The quick or dynamic opening and closing of the yaws allows to manipulate the tissue.
Ratchet: Intermittent pull and or push motions of a cable/rod converted in a rotary motion by a ratchet. The rotary motion can than be used to drive a spindle or to actuate a gear box.
Dual pitch rotatable threaded support (lead screw): one part of the rotatable threaded support is provided with a threaded region of a first pitch and used to control the opening and closing of the jaws; another part of the rotatable threaded support is provided with a threaded region of a second pitch another pitch is used advance the sled.
Cardan: a cardan may be provided to transmit the forces e.g. rotational forces.

The fusing head may be provided with a slidable cutting blade. The cutting blade may be actuated by a rotatable threaded support (lead screw), electric motor, piezoelectro motor, drive shaft, a block and tackle mechanism, or a pushing rod. The cutting blade may be attached in relation to e.g. a slidable constraining member, or sled (see later below).

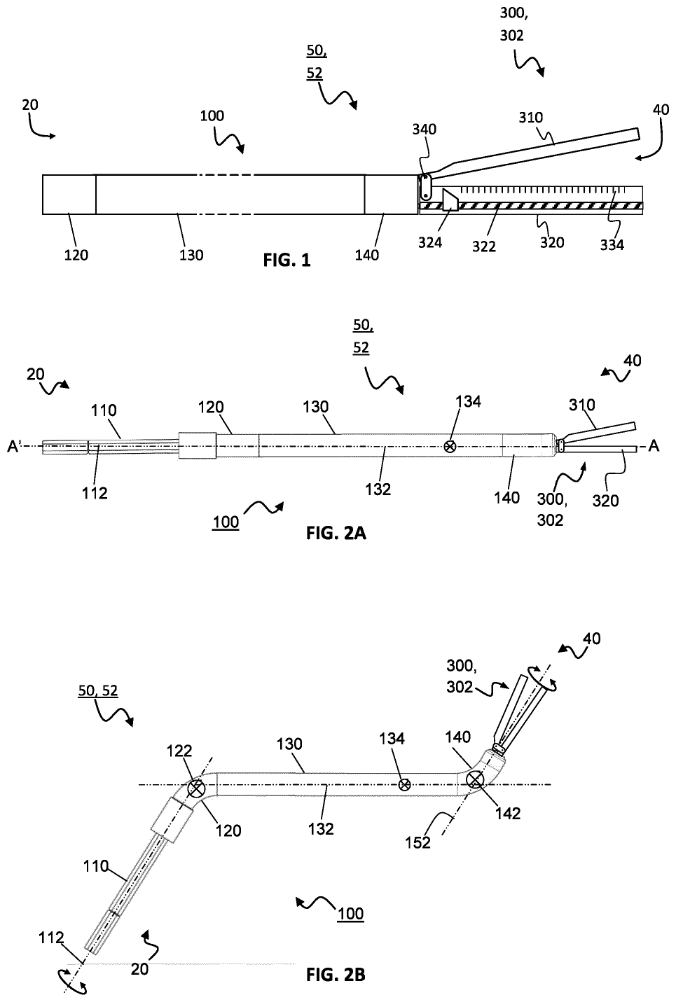

The fusing head may be a stapler head. The stapler head comprises a set of jaws moveable between an open and closed position, configured to receive one or more objects for stapling in the open configured. Application of force brings the jaws to a closed position, dispenses staples through the one or more objects, and bends the staple. An example of a stapler head (300) is shown in FIG. 1.

The stapler head has a jointed end and an opening end. The staple cartridge jaw and anvil jaw are connected at the jointed end to one or more joints allowing movement of the jaws between the open and closed position. The jointed end may disposed distal to an opening end of the jaws. The jointed end may disposed proximal to an opening end of the jaws.

The stapler head comprises a staple cartridge jaw configured to support a staple cartridge holding a plurality of surgical staples and an anvil jaw disposed with an anvil plate, the anvil jaw or staple cartridge jaw being moveable with respect to the other jaw between an open and closed position. The anvil plate comprises one or more rows of anvil clinching pockets corresponding to rows of staple-containing slots.

The staple cartridge comprises a plurality of staple-containing slots. Each slot is disposed with a staple. Staple cartridge may comprise a body portion having one or more, preferably at least 2 longitudinally extending rows of staple containing slots. The longitudinally extending row extend in a proximal to distal direction. The staple cartridge may be non-removeably or dismountably attached to the staple cartridge jaw. Each staple-containing slot may be disposed with a pusher element that deploys the staple out from the slot and towards the corresponding anvil clinching pockets.

The pusher element may be actuated by a sled member—a carriage slidable in relation to the staple cartridge jaw configured to drive staples and out of respective staple-containing slots and into anvil plate via the pusher element. An example of a sled member (324) is shown in FIGS. 1, and 3 to 6.

The sled member is disposed in slidable relation to the staple cartridge jaw. It may have a single axis of movement. Movement of the sled member is controllable.

The sled member may be mounted in relation to a rotatable threaded support extending through staple cartridge jaw. The rotatable threaded support may be a lead screw. The rotatable threaded support may be rotatably supported at its distal end, for instance by a pin extending proximally from the threaded support. The pin may be threaded into a hole formed in distal tip of staple cartridge jaw or staple cartridge. Rotation of threaded support causes sled member to move longitudinally within the staple cartridge jaw. The rotatable threaded support may directly drive the sled e.g. the sled is threaded or is connected to a threaded carriage. The rotatable threaded support may directly drive the slidable constraining member which abuts the sled so causing it to advance. The rotatable threaded support may be disposed in the staple cartridge jaw, in particular in the staple cartridge.

The sled member may be propelled by a linear piezoelectric motor. The sled member may be advanced by pushing/pulling motion of the drive shaft.

The sled member may be provided with a base having one or two vertically extending and tapered sides and extending vertically from base. Tapered sides and are provided to drive staples with into anvil plate. The taped sides taper off in a distal direction. In order to drive staples and upwardly through staple-containing slots, the staple cartridge may be provided with pusher elements positioned between staples. As the sled member moves distally relative to staple cartridge, the tapered sides of the sled member engage the pusher elements to drive staples out of staple-containing slots through the one or more objects to be stapled and into anvil plate.

The fusing head may be a sealing head. The sealing head is a device that seals object together by the application of energy seals the one or more object using heat. Heat may be provided to by bipolar electricity, by electrical welding or by piezo welding. The vessel sealer head comprises a set of jaws moveable between an open and closed position, configured to receive one or more objects for sealing in the open configured. Application of force brings the jaws to a closed position. In the close position, sealing energy (e.g. electricity, ultrasound, heat) is applied to the one or more objects.

The sealing head has a jointed end and an opening end. The jaws are connected at the jointed end to one or more joints allowing movement of the jaws between the open and closed position. The jointed end may disposed distal to an opening end of the jaws. The jointed end may disposed proximal to an opening end of the jaws.

The fusing head may be actuatable by means of a drive shaft that is at least partially flexible. It may be flexible along its entire length. The drive shaft is disposed within a lumen of the steerable instrument. The drive shaft is flexible at least in a region corresponding to the bendable distal part of the steerable instrument. The drive shaft is flexible at least in a region corresponding to the bendable proximal part of the steerable instrument. The drive shaft may be flexible or rigid in a region corresponding to the shaft of the steerable instrument. A drive shaft that is a combination of a rigid part corresponding to the shaft and flexible parts corresponding to the BDP and BPP has an advantage that the diameter of the drive shaft can be increased the in rigid part. The rigid part in the shaft exhibit greater torque resistant.

The drive shaft may be configured for the transmission of torque from the proximal end to the distal end of the steerable instrument. The drive shaft may alternatively or additionally be configured for the transmission of pulling and/or pushing forces from the proximal end to the distal end of the steerable instrument.

At the proximal end, the drive shaft may be attached to an actuating unit, such as a motor, lever or slider. At the proximal end, the drive shaft may be provided with an attachment element such as a loop, sphere, keyshaft, rotor for coupling with an actuating element.

The drive shaft in at least the flexible parts may be, for instance:
A torque cable or torque rope. This is solid (not hollow). The surface may be PTFE coated or provided with another lubricity surface. Preferably a multi-layer, multi-filar, stranded flexible shaft with high bi-directional torque transmission properties with minimal backlash or lag.
Torque tube: This is hollow. A hollow wire rope allows one or more additional cables or tubes to be installed within its lumen.
Hypotube: this is made by smart laser cutting patterns (e.g. slotted or spiral) in a hollow tube. The shaft performance characteristics such as flexibility, torque response and axial stiffness can be modified. The tube can be further protected with a jacket such as e.g. a shrink tube.
Superelastic drive shafts e.g. Nitinol wires, rods or tubes.
Polymers with or without a woven metal mesh (braid) to increase torqueability.
A plurality of articulated segments (described later below)

The rigid part of the drive shaft where present may be made from a stiff rod (polymer or metal).

The drive shaft may take one of the following configurations:
Solid such as a torque cable or a Nitinol wire. A solid form is able to transmit rotary or push/pull motions.
Hollow such as a torque tube or hypotube. A solid form could is also able transmit rotary motion and push/pull motions.
Hollow outer tube with inner slidable and/or rotatable inner : this allows up to four functions. The angulation difference could provide a fifth function.
Hollow outer tube, a lumen thereof disposed with a hollow inner tube, a the hollow inner tube disposed with a solid cable. The outer tube, inner tube and cable may be slidable and/or rotatable relative to each other.
Provided with an optional protective cover such as e.g. a polyimide tube. Such protective cover prevents small particles from being generated by friction between the irregular torque cable and a lumen wall which could leave the instrument.

Where the fusing head is a stapler head, the drive shaft at the distal end may be connected to the stapler head, in particular to the jaw actuating mechanism so as to close the jaws, and also to deploy staples.

The drive shaft may be attached at a distal end to the stapler head, for instance to a rotatable threaded support.

In particular, in order to rotate threaded support and thus translate sled through the staple cartridge jaw, a proximal end of threaded support may be connected to the drive shaft . Such drive shaft is configured for the transmission of torque. A distal end of the drive shaft may be attached in fixed rotational relation to a proximal end of the threaded support. Thus, rotation of drive shaft rotates the threaded support to translate sled through staple cartridge jaw, thereby deploying staples. A proximal end of the drive shaft may be attached to the actuating unit or motor unit. Drive shaft rotates the drive shaft.

A drive shaft that is a torque tube may rotate the threaded support that moves a constraining of a stapler head and provides a strong closing of the jaws. A pulling cable inside the torque tube allows for quick opening and closing of the jaws as to manipulate the tissue. At the stapler head there may be a small gearbox between the torque tube and a threaded support e.g. two cog-wheels so to further reduce the rotation speed of the spindle.

Where the fusing head is a sealing head, the drive shaft is attached at a distal end to the sealing head, for instance to a jaw actuating mechanism.

In a preferred aspect, the BDP is configured to bend along a curve and contains at least 3 (e.g. 3, 4, 5, 6 or more) tandemly arranged joints. Each joint is configured to locally bend the drive shaft disposed within a lumen of the BDP by an angle equal or less than 30°. In a preferred aspect, each LM guide in the BDP is configured to locally bend the drive shaft disposed within a lumen of the BDP by an angle equal or less than 30°. By restricting the local bend angle, and providing a plurality of joints, the drive shaft is able to transmit torque even when the BDP is bent at angles approaching 90° or more with respect to the shaft. Furthermore, kinking of the drive shaft is avoided compared with instruments that employ a single hinge type joints.

In a preferred aspect, the BPP is configured to bend along a curve and contains at least 2 (e.g. 2, 3, 4, 5, 6 or more) tandemly arranged joints. Each joint is configured to locally bend the drive shaft disposed within a lumen of the BDP by an angle equal or less than 30°. In a preferred aspect, each LM guide in the BPP is configured to locally bend the drive shaft disposed within a lumen of the BPP by an angle equal or less than 30°. By restricting the local bend angle, and providing a plurality of joints, the drive shaft is able to transmit torque even when the BPP is bent at angles approaching 90° or more with respect to the shaft. Furthermore, kinking of the drive shaft is avoided compared with instruments that employ a single hinge type joints.

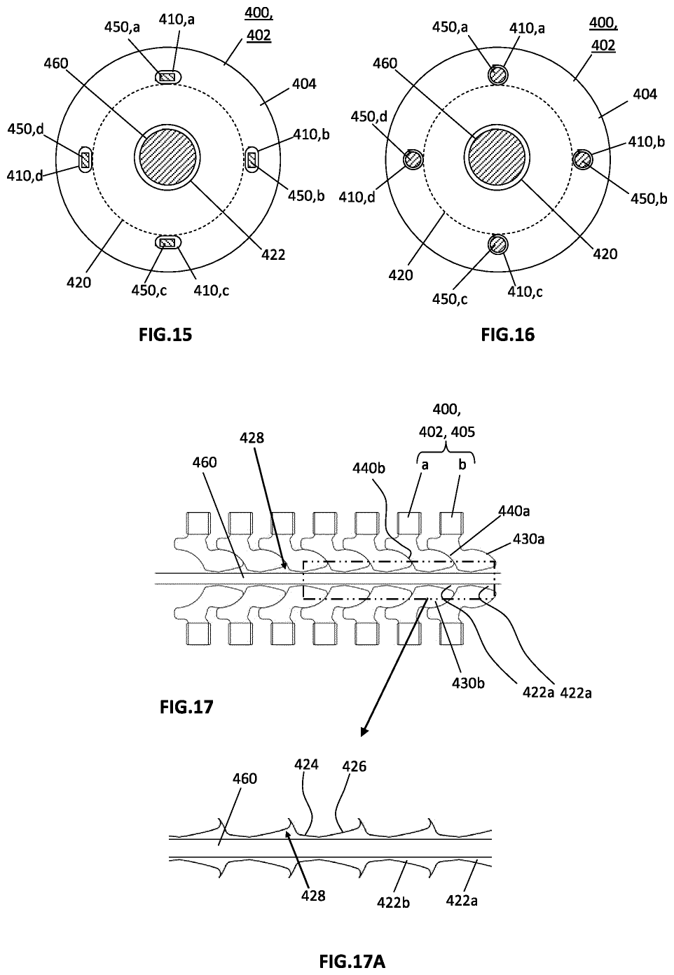

In a preferred aspect, drive shaft is disposed in a lumen of each joint of the tandemly arranged joints disposed in the BDP. The tandemly arranged lumens form an effectively continuous lumen through the BDP, which acts a bearing for the drive shaft. The transverse profile of the drive shaft has a smaller dimension than the transverse profile of the lumen. The transverse profile refers to a cross-sectional profile perpendicular to an axial (A-A′) direction, for instance the respective profiles of the drive shaft (460) and lumen (420) as shown in FIGS. 15 and 16. The drive shaft may have at least a close running engineering fit to the lumen of the joint. The transverse profile of the drive shaft may be up to 99.8% of the transverse profile of the lumen of the joint. To accommodate tolerances, the transverse profile of the drive shaft may be 25% to 99.8% of the transverse profile of the lumen of the joint in the BDP. The size of the respective profiles are determined by area of the profiles. Restricting clearance of the drive shaft within the lumen reduces play or backlash for actuation of the fusing head. It also prevents or reduces twisting of the drive shaft. The transverse profile of the drive shaft and lumen may both be circular. Restricting clearance between the drive shaft and lumen wall places the drive shaft within a centre line of the BDP; as the central line remains of a constant length even during bending there is a reduction in cross-talk between directional movements of the distal tip and the forces applied to the drive shaft.

In a preferred aspect, drive shaft is disposed in a lumen of each joint of the tandemly arranged joints disposed in the BPP. The tandemly arranged lumens form an effectively continuous lumen through the BPP, which acts a bearing for the drive shaft. The transverse profile of the drive shaft has a smaller dimension than the transverse profile of the lumen. The drive shaft may have at least a close running engineering fit to the lumen of the joint. The transverse profile of the drive shaft may be up to 99.8% of the transverse profile of the lumen of the joint. To accommodate tolerances, the transverse profile of the drive shaft may be 25% to 99.8% of the transverse profile of the lumen of the joint in the BPP. The size of the respective profiles are determined by area of the profiles. Restricting clearance of the drive shaft within the lumen reduces play or backlash for actuation of the fusing device head. It also prevents or reduces twisting of the drive shaft. The transverse profile of the drive shaft and lumen may both be circular. Restricting clearance between the drive shaft and lumen wall places the drive shaft within a centre line of the BPP; as the central line remains of a constant length even during bending there is a reduction in cross-talk between directional movements of the distal tip and the forces applied to the drive shaft.

The lumen thus acts as an outer sheath of a Bowden cable. It serves as an uncompressible sheath that cannot buckle. In this way more economic use is made of the available cross-sectional area of the lumen. It is still foreseen that a thin outer sheath is disposed over the drive shaft as a protective guide or to reduce friction or to prevent creation of fine debris.

The drive shaft at the proximal end may be connected to a dismountable instrument rotary coupling i.e. a body in rotation relative to the BPP configured to couple with a complementary dismountable rotary coupling in a robotic arm or in a dismountable handle. The complementary dismountable rotary coupling may be connected to a motor unit including a motor shaft for engagement with a gearbox. As motor is activated, rotation of motor shaft causes rotation of gears (not shown) of gearbox and thus rotation of drive shaft via the rotary coupling.

The drive shaft at the proximal end may be connected directly to a motor or motor unit, for instance in the case of a non-dismountable handle.

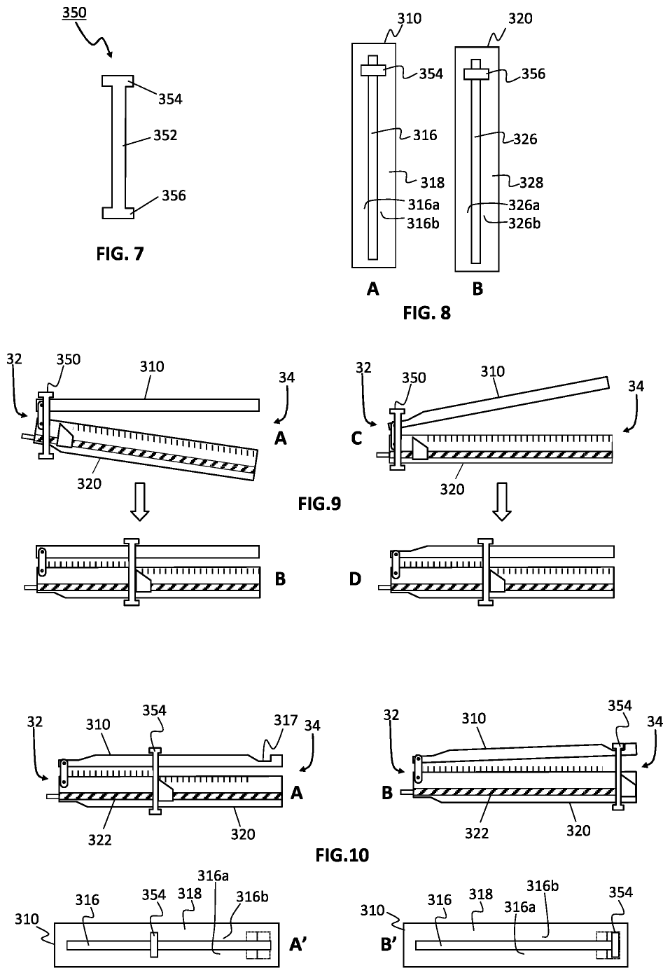

The stapler head may further comprising a slidable constraining member configured to move an opening end of the anvil jaw or staple cartridge jaw closer to the other jaw by constraining a distance between the jaws. Exemplary slidable constraining members are shown in in FIGS. 7 to 10.

The constraining member is slidable with respect to the stapler head. The constraining member is slidable with respect to the anvil jaw or staple cartridge jaw. Movement of the constraining member in a direction away from a jointed end moves an opening end of the anvil jaw or staple cartridge jaw closer to the other jaw.

The constraining member may be disposed in co-operation with the rotatable threaded support such that rotation of the rotatable threaded support advances the constraining member to actuate movement of an opening end of the anvil jaw or staple cartridge jaw closer to the other jaw. The constraining member may be attached in fixed (rotational and displaceable) relation to the sled member.

The constraining member may have a neutral position and an engaged position with respect to the anvil jaw and/or staple cartridge jaw, and is slidable between them. The neutral position of the constraining member corresponds to an open position of the jaws. The engaged position of the constraining member corresponds to a closed position of the jaws. It is noted that the neutral and engaged positions may correspond to a point or region on the anvil jaw or staple cartridge jaw. The neutral position may be disposed towards the jointed end; the constraining member may not be engaged with the anvil jaw or staple cartridge jaw such that the jaws are in an open position. The engaged position may be disposed away from the neutral position and towards the opening end of the jaws; the constraining member is engaged with the anvil jaw and staple cartridge jaw such that the jaws are in a closed position.

The constraining member may comprise a spacing beam flanked by a pair of stop members, where the spacing beam is disposed in a slot of a body present in each of the anvil jaw and staple cartridge jaw and each stop member abuts the slot edges thereby retaining the anvil jaw and staple cartridge jaw together at a distance determined by spacing beam when the constraining member is in an engaged position.

The anvil jaw and/or staple cartridge jaw may be disposed with a cam between the open and closed position. It allows a smooth movement of the constraining member between the neutral and engages positions. Forces applied by the constraining member sliding over the cam are transferred to the jaws causing them to close.

The spacing beam is rigid. The spacing beam is non-expandable. The distance of the spacing beam between the stop members is fixed.

As shown, for instance, in FIGS. 10A and 10B the slot (316) may be disposed with a position discrete recess (317) towards an opening end (34) of the slot (316) for receiving the stop member (354) such that engagement in the recess (317) by the stop member (354) increases the distance between the anvil jaw (310) and staple cartridge jaw (320) allowing release of the object. The recess may be formed in a body (318) of the anvil jaw (310).

Alternatively, the recess may be replaced with a position discrete aperture disposed towards an opening end of the slot for passage of the stop member therethrough such that passage through the aperture by the stop member increases the distance between the anvil jaw and staple cartridge jaw allowing release of the object. The aperture may be formed in a body of the anvil jaw.

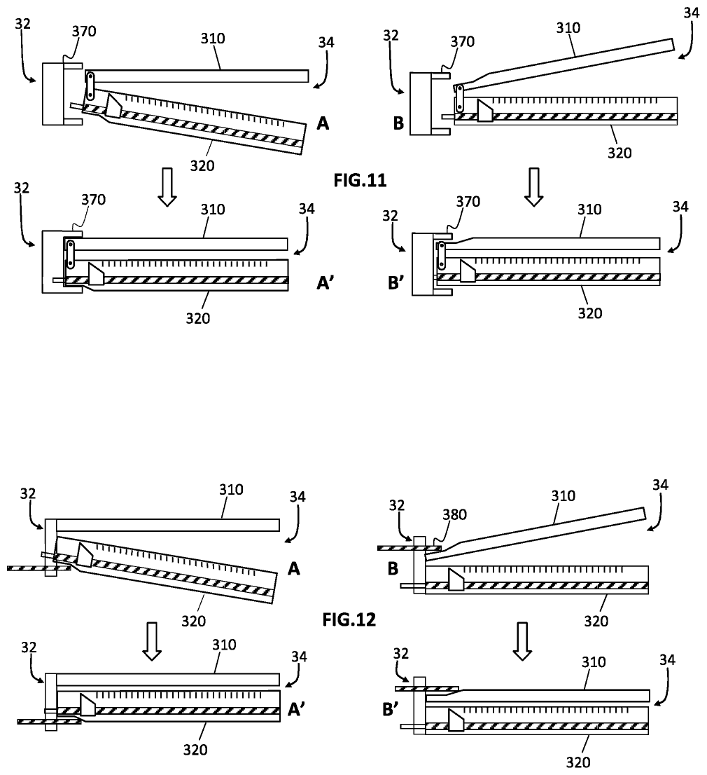

The stapler head may further comprising a constraining collar configured to move an opening end of the anvil jaw or staple cartridge jaw closer to the other jaw. Examples of a constraining collar (370) are given in FIG. 11.

The constraining collar comprises a passage configured to receive and couple with a joined end of the jaws, thereby moving an opening end of the anvil jaw or staple cartridge jaw closer to the other jaw.

The constraining collar may be slidable with respect to the stapler head. The stapler head may be slidable with respect to the constraining collar. The constraining collar may be slidable with respect to the anvil jaw or staple cartridge jaw. The anvil jaw or staple cartridge jaw may be slidable with respect to the constraining collar. Movement that brings to jointed end into constraining collar passage moves an opening end of the anvil jaw or staple cartridge jaw closer to the other jaw.

The constraining collar and jointed end of the jaws may mutually adopt a neutral position or an engaged position and can transition between them. Preferably the jointed end of the jaws is slidable between the neutral position or an engaged position. The neutral position of the constraining collar and jointed end combination corresponds to an open position of the jaws. The engaged position of the constraining collar and jointed end combination corresponds to a closed position of the jaws. It is noted that the neutral and engaged positions may correspond to a point or region on the anvil jaw or staple cartridge jaw. The neutral position may be disposed towards the jointed end; the constraining collar may not be engaged with the anvil jaw or staple cartridge jaw such that the jaws are in an open position. The engaged position may be disposed away from the neutral position and towards the opening end of the jaws; the constraining collar is engaged with the anvil jaw and staple cartridge jaw such that the jaws are in a closed position.

The constraining collar may comprise a rigid body disposed with a passage configured to receive and couple with a joined end of the jaws. The constraining collar is rigid. The constraining collar is non-expandable. The size of the constraining collar passage is fixed.

Movement of the joined end of the jaws into the passage applies a closing force to one or both jaws. The anvil jaw and/or staple cartridge jaw may be disposed with a cam between the open and closed position. It allows a smooth movement of the jaws with respect to the constraining collar between the neutral and engages positions. Forces applied by the cam sliding relative to the constraining collar are transferred to the jaws causing them to close.

The stapler head may further comprising a closing screw configured to contact and upon rotation advance relative to, the anvil jaw or staple cartridge jaw, thereby moving an opening end (34) of the anvil jaw or staple cartridge jaw closer to the other jaw. An example of a closing screw is shown in FIGS. 12A, A′ and B, B′.

The staple cartridge jaw and anvil jaw may be connected at a jointed end to one or more joints allowing movement of the jaws between the open and closed position, wherein the jointed end is disposed proximal to an opening end of the jaws. The staple cartridge jaw or staple cartridge may comprises a rotatable threaded support and a sled member whereby rotation of the rotatable threaded support advances the sled member in the distal direction to deploys staples from the staple cartridge and into the anvil plate.

The staple cartridge jaw and anvil jaw may be connected at a jointed end to one or more joints allowing movement of the jaws between the open and closed position, wherein the jointed end is disposed distal to an opening end of the jaws, as shown, for instance in FIG. 4. The staple cartridge jaw or staple cartridge comprises rotatable threaded support and a sled member whereby rotation of the rotatable threaded support advances the sled member in the proximal direction to deploy staples from the staple cartridge and into the anvil plate. As mentioned this configuration allows for better visualtion of the object to be fused the object to be scooped into the jaws.

The staple cartridge jaw and/or staple cartridge, and/or the anvil jaw may each disposed with respective clamping members which co-operate in the closed position to clamp an object e.g. tissue captured between jaws (310, 320) of the stapler head (300). The clamped object may subsequently be cut with a knife disposed in fixed relation to the sled member. The staple cartridge jaw or staple cartridge tissue clamping member may comprise a blunt, sharpened or serrated longitudinal edge. The anvil jaw tissue clamping member may comprise a blunt, sharpened or serrated longitudinal edge.

The BDP is configured to move omni-directionally i.e. in any radial direction while the shaft is rotationally fixed. BDP is preferably configured to move in any radial direction (about 360° with respect to central longitudinal axis (A′-A) of the shaft part) while the shaft is axially-rotationally fixed. The BDP may be configured to bend along a curve. It might be distinguished from classical minimally invasive tools in that it may be absent of revolute joints. The movement response of the BDP may be:
a change in degree of bending within a “bending plane” that is a plane parallel to and contacting a central longitudinal axis (A-A′) of and extending from the shaft,
a change direction of the bend ; it amounts to a change in direction of the bending plane around the shaft central longitudinal axis (A-A′) when the BDP lies along said bending plane .

Similarly, the BPP, is configured to move omni-directionally i.e. in any radial direction while the shaft is rotationally fixed. BPP is preferably configured to move in any radial direction (about 360° with respect to central longitudinal axis (A′-A) of the shaft part) while the shaft is axially-rotationally fixed. The BPP may configured to bend along a curve. It might be distinguished from classical minimally invasive tools in that may be absent of revolute joints.

The movement response of the BPP may be:
a change in degree of bending within a “bending plane” that is a plane parallel to and contacting a central longitudinal axis of and extending from the shaft,
a change direction of the bend i.e. of the distal tip or end effector; it amounts to a change in direction of the bending plane around the shaft central longitudinal axis (A-A′) when the BPP lies along said bending plane.

The combination of movements of the steerable instrument facilitates a rotation of BPP at its tip or of the stapler head while the BPP is in a bent position that is transmitted via a rotation of the shaft to the BDP that causes rotation of the BDP tip or fusing head while the BDP is in a bent position. With such rotation of the tip or of the fusing device head, the direction of the bending plane can be maintained constant.

The combination of the movement of steerable instrument further facilitates a change in direction of the BDP tip or fusing device head while the shaft is in a fixed rotational position. With such movement, the bending plane rotates around the shaft central longitudinal axis (A-A′) while the shaft itself does not rotate.

The length of the central line of the steerable instrument may be constant when the BDP and BPP are both in a straight or bent configuration. In such a way that central line of the drive shaft coincides with the central line of the steerable instrument, the steerable tube takes on an extra function of the outer tube of a Bowden Cable assembly. When the drive shaft is being pulled, pushed or rotated, the steerable instrument will optimally take the reaction forces (due to column stiffness for pulling pushing and due to torque stiffness for rotation). When the steerable tube is being bent, no crosstalk exists between the actuating of the drive shaft and the bending (due to constant length central line). When the steerable instrument is being rotated about its axis (central line), the proximal end rotates exactly the same angle as the distal end. Therefore, no crosstalk exists between the rotation of the steerable instrument about its axis and the rotation of the drive shaft.

To control the BDP responsive to movements of the BPP or response to actuators, steering wires which are known as longitudinal members (LMs) are provided. The LM controls the BDP by pulling or pushing. The steerable instrument comprises a set of longitudinal members (LM) each having a proximal end and a distal end, arranged in a longitudinal direction around a fictive tube. A transverse profile of at least one LM may be circular. A transverse profile of at least one LM may demonstrate an anisotropic area moment of inertia; the transverse profile of the LM may have a square, rectangular, serif letter “I”, or circular segment profile, optionally wherein one or more of the profile corners are pointed or rounded-off. The transverse profile refers to a cross-sectional profile perpendicular to an axial (A-A′) direction. The presence of anisotropic moment of inertia in the LMs yields a steerable tube having a high torque stiffness. The LMs may be cut from a tube or provided as separate strands. With this arrangement, the tip (distal terminal end) of the BDP moves with equal ease in any direction i.e. there is no singularity. The movement response is proportion to the degree of actuation. An example of a transmission mechanism has been described in WO 2009/098244.

The steerable instrument may be disposed with an inner and/or outer tube configured to reduce bucking of the LMs. The inner and/or outer tube is flexible at least in the BDP and BPP. The outer tube made be a flexible tube that covers the LMs. The inner tube and/or outer tube may be a tube having a stiff part corresponding to the shaft and a flexible part corresponding to the BDP and BPP; the flexibility may be achieved by the presence of cuts in the stiff tube wall. The presence of an inner and/or outer tube contributes to a higher column stiffness. The improved column stiffness is evident when the BDP and BPP are in the straight and also in bent position. Even though the inner and/or outer tube's integrity is significantly affected by the longitudinal cuts, the column stiffness is hardly affected due to the extra elements that keep the longitudinal elements from buckling. These extra elements are the outer sheath and inner lining for a 3tube system. These extra elements can also be (polymer) links.

The BPP and BDP may each be provided with one or a plurality of tandemly arranged pivoting joints. Each pivoting joint allows movement in 2 degrees of freedom. Each joint preferably restricts axial rotation. Each pivoting joint may be made from two articulated joint parts (e.g. ball and socket). The pivoting joints may be formed by a set of articulated joint parts that are longitudinal member (LM) guides present in each of the BPP and BDP. The term articulated part and LM guide may be used interchangeably herein.

The pivoting joints may be formed by a set of longitudinal member (LM) guides present in each of the BPP and BDP. An LM guide comprises a body having a proximal side, a distal side and an outside edge, wherein the body of the LM guide comprises a set of channels arranged around a fictive tube. Each channel passes from the proximal side to the distal side of the body. Each channel is configured to retain an LM of a set of LMs in a fixed radial position around the fictive tube. Each channel thus constrains radial movement of a set of LM. Each channel may further be configured to provide a discrete constraining point to axially rotationally constrain an LM. A channel may have a transverse profile complementary to that of the LM. A channel may have a circular transverse profile. A channel may have a rectangular transverse profile. A channel may have a circular transverse profile. At least one or two of the LM guides in the set may be articulated LM guides tandemly arranged and are mutually articulated, giving rise to the pivoting joints thereby supporting bending of the LMs in the BPP and BDP.

An example of an LM guide (402) that is an articulated LM guide (405) is depicted in FIG. 14. The number of articulated LM guides in the BPP may be at least 1 or 2 (e.g. 2, 3, 4, 5, 6, 7, 8 or more), preferably at least 5; where there is at least 2, the BPP may bend along a curve. The number of articulated LM guides in the BDP may be at least 1 or 2 (e.g. 2, 3, 4, 5, 6, 7, 8 or more), preferably at least 5; where there are at least 2 LM guides, the BDP may bend along a curve. The articulated LM guides are in pairwise mutual contact through a pivot joint. The pivot joint may comprise a ball and socket joint, a flexible part, such as a rubber or silicone element, or a stack of spherical bodies. Two adjacent articulated LM guides give rise to 1 pivoting joint. Each articulated LM guide can be flanked on each side by another articulated LM guide. An arrangement of LMs and LM guides, and of a transmission mechanism for a steerable instrument have been described in WO 2016/030457 and WO 2016/091856, and are incorporated by reference herein.

In an alternative arrangement, the BPP and BDP may each be provided with a sleeve containing a plurality of arc shaped discrete slits each provided essentially perpendicular to a longitudinal axis of the BPP or BDP. Each slit may span an angle of around 150 to 210 deg. Advancing along the BPP or BDP, an orientation of a slit may change with respect to a previous slit. Preferably, each slit spans an angle of around 180 deg, and the orientation alternates between 0 and 180 deg advancing along the BPP or BDP. The sleeve supports to the LMs, while the slits allow bending of the BPP or BDP in any direction.

In an alternative arrangement, the BPP and BDP may each be provided with a plural of interconnected discs. Each disc is connected by a flexible spacer (e.g. smaller polymeric cylinder) disposed towards the centre the disc, and such carries the lumen The plurality of interconnected discs supports to the LMs, while the spacers allow bending of the BPP or BDP in any direction.

The bending angle of the BDP, which is an angle between a central axis (FIG. 2B, 152) of the distal tip or fusing device and of a central axis of the shaft (FIG. 2B, 132) may be a maximum of 180 deg, preferably of 100 deg, more preferably of 90 deg. The number of pivoting joints in the BDP may be at least 3. The number of articulated LM guides in the BDP may be at least 2. A pivoting joint may be configured to locally bend the drive shaft disposed within a lumen of the BDP by an angle (α) equal or less than the bend angle/ the number of pivoting joints. In a preferred aspect, each articulated LM guide in the BDP is configured to locally bend the drive shaft disposed within a lumen of the BDP by an angle (α) equal or less than bend angle/ the number of pivoting joints. The value of α may be 30 deg or less as mentioned above.

The bending angle of the BPP, which is an angle between a central axis (FIG. 2B, 112) of the proximal tip or connector and of a central axis of the shaft (FIG. 2B, 132) may be a maximum of 180 deg, preferably of 100 deg, more preferably of 90 deg. The number of pivoting joints in the BPP may be at least 3. The number of articulated LM guides in the BPP may be at least 2. A pivoting joint may be configured to locally bend the drive shaft disposed within a lumen of the BPP by an angle (α) equal or less than the bend angle/ the number of pivoting joints. In a preferred aspect, each articulated LM guide in the BPP is configured to locally bend the drive shaft disposed within a lumen of the BPP by an angle (α) equal or less than bend angle/ the number of pivoting joints. The value of α may be 30 deg or less as mentioned above.

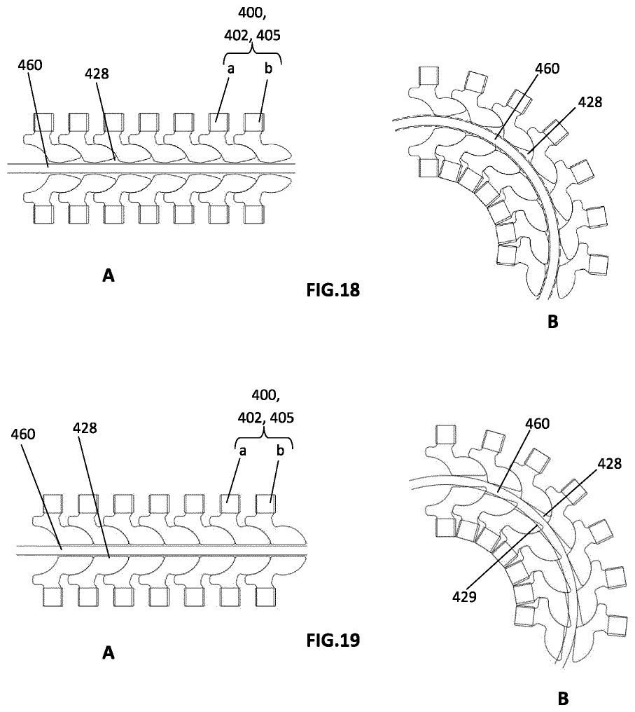

Each of the plurality of tandemly arranged pivoting joints may be provided with a central lumen for passage of the drive shaft. Each of the plurality of tandemly arranged articulated joints parts may be provided with a central lumen for passage of the drive shaft. The separate lumens of the articulated joints parts form an essentially continuous lumen in the DBP and BPP. Each separate lumen passes from a “ball” part to a “socket” of a pivoting joint, in particular when it is formed from articulated joints parts or articulated LM guides. Each lumen acts as a bearing that supports movement of the drive shaft. The lumen may be cylindrical. The wall of cylindrical lumen of each joint may be flared outwards at one or both ends so as to avoid gripping the drive shaft when the DBP and/or BPP is in a bent configuration. By flared it is means that a transverse profile of the cylindrical lumen gradually increases towards an end, for instance, towards a distal end or proximal end. Where the wall of cylindrical lumen of each joint is flared outwards at both ends, the result can be an “hourglass” type profile. The shape of the flared end can be any e.g. funnel shaped, trumpet shape, “diabolo” shaped, chamfered, rounded, conical, hyperboloid. As shown in FIGS. 18A and B, when each the lumen of each articulated joint part (LM guide (405)) is flared (424, 426) at both proximal and distal ends (FIGS. 18A), the bending (FIGS. 18B) places the flexible part of the drive shaft (460) in the centre of the lumen (420) and avoids biting or locking its movements. By contrast when the each the lumen of each articulated joint part (LM guide (405)) is has a uniform cylindrical from proximal to distal ends (FIGS. 19A), the bending (FIGS. 19B) causes the wall of the lumens to bite or lock against the drive shaft (460) thereby restricting its movements.

The drive shaft in at least the BDP and/or BPP may be formed from a plurality of articulated segments as shown, for instance, in FIGS. 21-23. Each segment is articulated with an adjacent segment via joint that allows bending but which still transmits rotation. The joint may allow only 2DOF of movement, and maintains the segments in fixed rotation relation to eachother. Examples of such a joint include a universal joint, cardan joint, axially rotationally constricted ball and socket joint.

In a preferred aspect, each segment of the drive shaft is provided such that it is aligned within a lumen of corresponding articulated joint part (e.g. within a lumen of an articulated LM guide) of the BDP or BPP. Axial sliding of the segment within an articulated joint part lumen may be constrained, for instance, by providing an annular groove around the body of the drive shaft segment that engages with a clip disposed in relation to the corresponding articulated joint part. The direction of the axis of rotation of each segment within an articulated joint part lumen may be constrained, for instance, by matching the outer diameter of the drive shaft segment with the diameter of the lumen of the articulated joint part.

The number of segments in the drive shaft in the region of the BDP may be the same as the number of articulated joint parts (e.g. articulated LM guides) in the BDP. The number of segments in the drive shaft in the region of the BPP may be the same as the number of articulated joint parts (e.g. articulated LM guides) in the BDP.

An advantage of a segmented drive shaft is that it can transfer more force. Most typical shafts have property that their flexibility is a function of its diameter; a cable shaft becomes stiffer with increasing diameter. The stiffness of the drive shaft, determines the minimum bending radius that shaft can be subjected to without being damaged due to excessive strain. Violating yield strength implies limited durability and violating ultimate strength and catastrophical failure.

By employing the segmented drive shaft described, a change in angle of the shaft can be implemented without bending or internal strain in the drive shaft due to this bending. The available material strength can be employed in favour of the actuation of the drive shaft. By using a plurality of articulated segments, a limit to the bending angle of a single joint can be overcome. For instance, universal joint has a maximum angle for the fluid transmission of torque. By segmentation, bending angles of 180 deg can be achieved which still allow the transmission of rotational forces.

Realising, for instance, a total of 90 degrees bending angle of a BPP or BDP, using 6 tandemly arranged drive shaft segments therein giving rise to 5 articulated joints, would allows a maximum angle of 90/5 (90/n) or 18 degrees maximum per articulated joint. This lower angle allows the articulated joint to function fluently, with a nearly constant 1:1 angle transmission.

Providing every articulated joint part (e.g. articulated LM guide) with a drive shaft segment allows each drive shaft segment to be axially aligned within its own lumen. The drive shaft segment may be constrained to prevent or reduce sliding and/or to prevent or reduce changes to its axial direction relative to the lumen. Preferable, the axis of rotation of the drive shaft segment coincides with a central axis of the lumen. Constraining can prevent the drive shaft articulated segments from spiralling and locking up.

Preferably a centre point of articulation between a pair of articulated joint parts (e.g. articulated LM guide) coincides with a centre point of articulation between a pair of drive shaft segments.

A drive shaft articulated segment may have an essentially cylindrical body. A drive shaft articulated segment may have a spherical body (see for instance, FIG. 24). In such example, the spherical drive shaft articulated segment can also provide the joint between adjacent articulated joint parts (e.g. articulated LM guide). Synchronised rotation between adjacent spherical segments can realised through a philips-type coupling as seen in a cross-head screw driver.

#SI Examples of Suitable Steerable Instruments in the Art

The steerable instrument may be that described in, for instance, WO 2009/098244, WO 2016/030457, WO 2016/091857, WO 2016/091858.

The steerable fusing device may be an engineering tool, industrial tool, or surgical instrument, having use for any type of remote fusing (e.g. stapling or sealing) activity. The steerable stapler may be a surgical instrument, such as, for instance, a minimally invasive surgical instrument.

The minimally invasive instrument typically, but not necessarily enters the body via a trocar—a tube-like port inserted into an incision. The trocar is configured to receive the shaft of the steerable instrument; it is provided with a trocar passage into which the steerable instrument can axially slide and rotate, to support the steerable instrument allowing axial (A-A′) displacements and also to provide a fulcrum point to change direction of the steerable instrument. A trocar is known in the art. Where the trocar can pivot freely around the incision, so the steerable instrument can pivot around the fulcrum zone in concert with the trocar.

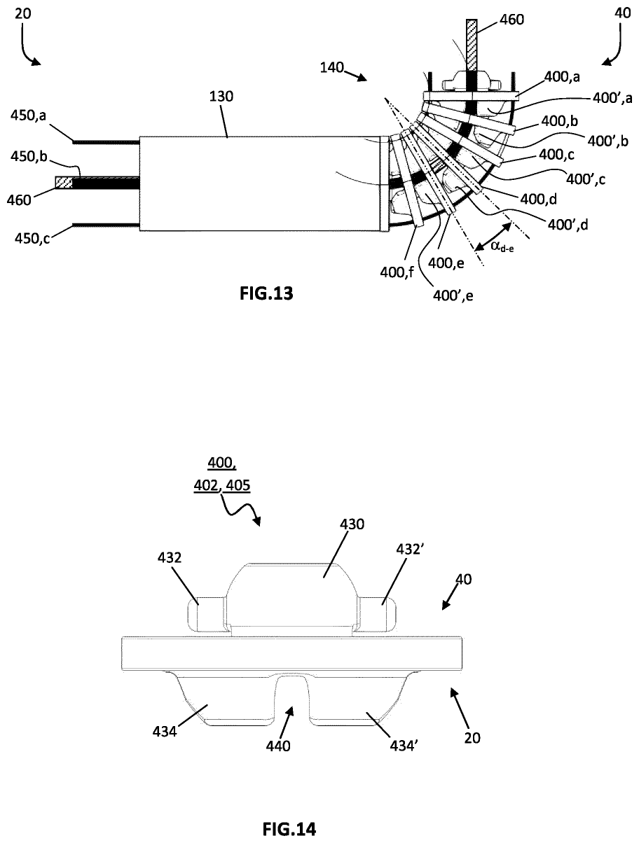

The steerable instrument may comprise a first and second BPP tandemly arranged and that controls movement of a first and second BDP respectively tandemly arranged, as described for instance in WO 2009/098244 (see FIG. 13A and 13B therein). In such case, the connector attached to the outer most (first) BPP controls movement of the outer most (first) BDP in the same way as described herein, and is attachable to a robotic arm. The second (inner most) BPP controls movement of the second (inner most) BDP; once the desired position of second (inner most) BDP is met, the position of the second (inner most) BPP is locked using an external clamp. Alternatively, the position of second (inner most) BPP may be controlled using an index mechanism that allows selection from a plurality of fixed discrete positions.

A robotic arm comprises a base end, an effector end and a plurality of intervening linkages connected by joints, wherein the arrangement of links and joints provides at least 6 degrees of freedom of movement to the effector end. The joints are actuatable, typically by motors, hydraulics, or pneumatics allowing control of the position and direction of the effector end by electronic signals. Each joint, also known as a kinematic pair, may offer 1 or 2 degrees of freedom (DOF) of movement, preferably 1 DOF. A joint may be a revolute or prismatic joint. A revolute joint has one degree of freedom of movement that is rotational. A prismatic joint has one degree of freedom of movement that is a linear displacement i.e. slidable. Typically a robotic arm comprises 6 joints each having 1 DOF to generate 6 DOF of movement to the effector end. Where a robot arm contains more than 6 joints, the position and direction of the effector can be attained using a plurality of different combinations of joint positions, offering redundancy that is useful for instance where the path of the robotic arm is restricted.

When the last joint is mentioned herein, it refers to the joint that is a kinematic pair of the kinematic chain at the effector end that would attach to the fitting. The last two joints refer to (1) the last joint and (2) the joint that is a kinematic pair of the kinematic chain attached to the last joint and disposed towards the base end of the robotic arm. The last three joints refer to (1) the last joint and (2) the joint that is a kinematic pair of the kinematic chain attached to the last joint and disposed towards the base end of the robotic arm, and (3) the joint that is a kinematic pair of the kinematic chain attached to joint (2) and disposed towards the base end of the robotic arm. The joints include any integrated into a robotic arm unit, and any joints added by way of an adapter added to the effector end of the robotic arm unit.

The robotic arm may be commercially provided, for instance, as manufactured by Kuka, Fanuc, ABB or may be an adapted commercially available robotic arm. An adaptation to an existing robotic arm includes, for instance, a replacement of one or more joints or linkage, or an addition of one or more controllably degrees of freedom using an adapter attached to the effector end thereby creating a new effector end.

The effector end is provided with a fitting for dismountable attachment to the connector. The fitting may be a standard fitting such as already provided by the robotic arm, or may customised according to the parameters of the connector.

A moveable member may be provided, wherein the base end (230) of the robot arm (200) is attached to the moveable member, and wherein the position of the moveable member is adjustable (displaceable in 1 or more directions), and optionally the angle of the moveable member is adjustable (rotatable in 1 or more directions).

The moveable member is comprised in a (motorised) gantry, a (motorised) trolley, or a further robotic arm.

Further provided herein is a system comprising the steerable instrument as described herein, and the robotic arm.

Further provided herein is a method of controlling a robotic arm as described herein to move an attached steerable stapler as described herein, which method effects movements of steerable instrument and stapler head that include:
rotation of the shaft around the fulcrum zone,
rotation of the shaft axially (A-A′),
displacement of the shaft axially,
bending of the bendable distal part, and
rotation of the steerable stapler when the bendable distal part is in a bent position.

According to the method movements of steerable instrument may be responsive to a manual input unit.

Movements of the steerable instrument responsive to a manual input unit may be determined using a (mathematical) model of the steerable instrument.

Further provided is a computing device or system configured for performing the method described herein.

Further provided is a computer program or computer program product having instructions which when executed by a computing device or system cause the computing device or system to perform the method described herein.

Further provided is a computer readable medium having stored thereon a computer program as described herein.

Further provided is a computer readable medium having stored thereon instructions which when executed by a computing device or system cause the computing device or system to perform the method as described herein.

Further provided is a data stream which is representative of a computer program or computer program product as described herein.

FIGS. 1 is an illustration of a steerable fusing device (50) that is a steerable stapler (52) as described herein. It has a proximal end (20) and a distal (40) end. The same reference sign for distal end (40) and proximal end (20) is applied herein also to other components of the steerable stapler (52) including the steerable instrument (100) and stapler head (302). The steerable stapler (52) comprises a steerable instrument (100). The steerable instrument (100) has a proximal end (20) and a distal (40) end and comprises a shaft (130), a bendable proximal part, BPP (120) and a bendable distal part, BDP (140). A fusing head that is a stapler head (302) is attached in fixed rotational relation to the distal terminal end (40) of the BDP (140). The stapler head (302) comprises a staple cartridge jaw (320) configured to support a staple cartridge holding a plurality of surgical staples (334) and an anvil jaw (310) disposed with an anvil plate, the anvil jaw (310) or staple cartridge jaw (320) being connected by a joint (340), and the anvil jaw (310) is depicted as moveable with respect to the staple cartridge jaw (320) between an open and closed position. The staple cartridge jaw (320) comprises a rotatable threaded support (322) and a sled member (324) whereby rotation of the lead screw (322) advances the sled member (324) away from the jointed end (32) to deploy staples (334) from the staple cartridge and into the anvil plate.

FIG. 2A is a further illustration of a steerable stapler (52) of FIG. 1. It has a proximal end (20) and a distal (40) end. The steerable stapler (52) comprises a steerable instrument (100). The steerable instrument (100) has a proximal end (20) and a distal (40) end and comprises a shaft (130), a bendable proximal part, BPP (120) and a bendable distal part, BDP (140). A connector (110) configured for dismountable attachment to the robotic arm (200) is attached in fixed rotational relation to the proximal terminal end (20) of the BPP (120). A fusing head (300) that is a stapler head (302) is attached in fixed rotational relation to the distal terminal end (40) of the BDP (140). The stapler head (302) comprises the staple cartridge jaw (320) anvil jaw (310) disposed with an anvil plate. The shaft (130) pivots around a fulcrum zone (134). A central axis (132) of the shaft (130), an axis of rotation (112) of the connector (110), and an axis of rotation (152) of the stapler head (302) are depicted.

FIG. 2B shows the steerable stapler (52) of FIG. 2A wherein the BDP (140) is bent responsive to a bending of the BPP (120), and the direction of the stapler head (302) diverges from the direction of the shaft (130)

In FIG. 2A, the BPP (120) and a BDP (140) are straight; an axis of rotation (152) of the end effector (150), a central longitudinal axis (132) of the shaft (130), and an axis of rotation (112) of the connector (110) are mutually coaxial. In FIG. 2B, the BDP (140) is bent responsive to bending of the BPP (120), and the end effector (150) is rotatable around its (axial) axis of rotation (152) when the BDP (140) is in a bent position (relative to the shaft) by a complementary rotation of the connector (110) around its (axial) axis of rotation (112). Axes of rotation (112) of the BPP (120) at different directions intersect at a zone of motion (122) along the BPP (120). In this figure, the BDP (140) bends along a curve, and not around a revolute joint; axes of rotation (112) of the BDP (140) at different directions intersect at a zone of motion (142) along the BDP (140).

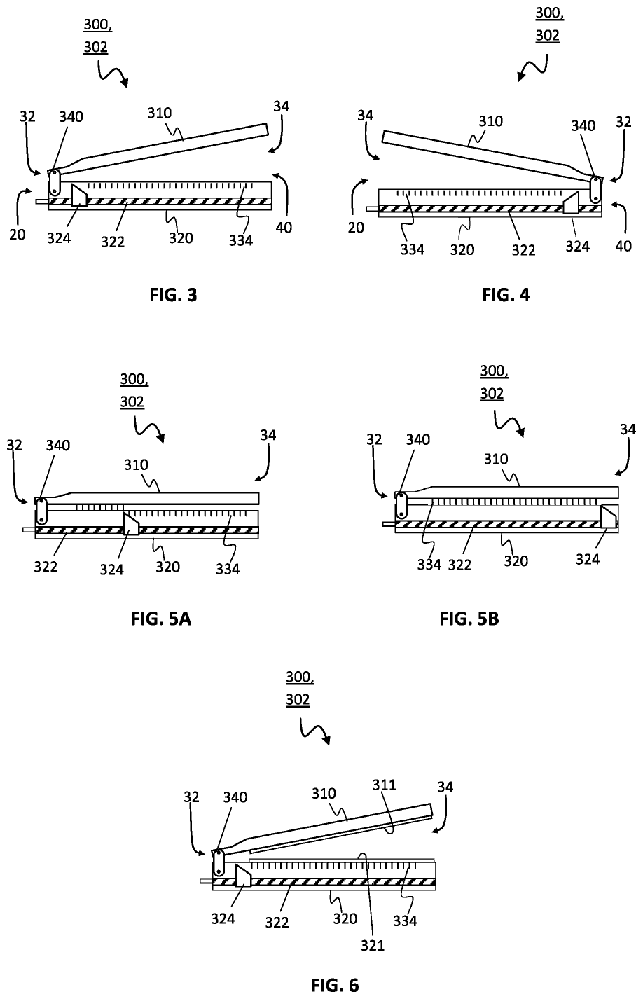

FIGS. 3 and 4 illustrate a stapler head (302) as described herein containing the same or similar features as the stapler head (302) of FIG. 1. Shown are also the jointed end (32) and opening end (34) of the stapler head (302). In FIG. 3 the jointed end (32) is oriented towards the proximal end (20) and the opening end (34) is oriented towards the distal end (40). Hence the sled member (324) advances in a distal (40) direction to deploy staples (334). In FIG. 4 the jointed end (32) is oriented towards the distal end (40) and the opening end (34) is oriented towards the proximal end (30). Hence the sled member (324) advances in a proximal (20) direction to deploy staples (334).

FIGS. 5A and 5B depict a stapler head (302) as described herein in the closed configuration containing the same or similar features as the stapler head (302) of FIG. 1. In FIG. 5A the sled member (324) is partially advanced in towards the opening end (34) deploying staples (334) against an anvil plate (not shown) of the anvil jaw (310). In FIG. 5B the sled member (324) is fully advanced in the opening end (34) direction.

FIG. 6 depicts a stapler head (302) as described herein in the open configuration containing the same or similar features as the stapler head (302) of FIG. 1. The staple cartridge jaw (320) is disposed with a tissue clamping member (321). The anvil jaw (310) is disposed with a separate tissue clamping member (311). Both tissue clamping members (311, 321) engage in the jaws-closed configuration to clamp the object being stapled.

FIG. 7 depicts a slidable constraining member (350) that is a serif i-beam. The constraining member (350) comprises a spacing beam (352) flanked by a pair of stop members (354, 356)

FIGS. 8A and 8B are upper and lower plan views of the stapler head (302). In FIG. 8A, showing a plan view of the anvil jaw (310) the spacing beam is disposed in a slot (316) of a body (318) present in the anvil jaw (310) and the stop member abuts the slot edges (316a, 316b). In FIG. 8B, showing a plan view of staple cartridge jaw (320) the spacing beam is disposed in a slot (326) of a body (328) present in the staple cartridge jaw (320) and the stop member abuts the slot edges (326a, 326b).

FIGS. 9A to 9D each depict a stapler head (302) as described herein containing the same or similar features as the stapler head (302) of FIG. 1, disposed with the constraining member (350) that is a serif I-beam. In FIGS. 9A and 9B, advancement of the constraining member (350) from the jointed end (32) (FIG. 9A) towards the opening end (34) (FIG. 9B) results in movement of the staple cartridge jaw (320) against the anvil jaw (310) thereby closing the jaws. In FIGS. 9C and 9D, advancement of the constraining member (350) from the jointed end (32) (FIG. 9C) towards the opening end (34) (FIG. 9D) results in movement of the anvil jaw (310) against the staple cartridge jaw (320) thereby closing the jaws.

FIGS. 10A and 10B each depict a stapler head (302) as described herein containing the same or similar features as the stapler head (302) of FIG. 1, disposed with the constraining member (350) that is a serif i-beam. FIGS. 10A′ and 10B′ each depict a corresponding plan view of the stapler heads (302) of FIGS. 10A and 10B from the top of the anvil jaw (310) containing the same or similar features as the anvil jaw (310) of FIG. 8A. The slot (316) is disposed with a position discrete recess (317) towards an opening end (34) of the slot (316) for receiving the stop member (354) such that engagement in the recess (317) by the stop member (354) increases the distance between the anvil jaw (310) and staple cartridge jaw (320) allowing release of the object. The recess may be formed in a body (318) of the anvil jaw (310).

FIGS. 11A, A′ and 11B, B′ each depict a stapler head (302) as described herein containing the same or similar features as the stapler head (302) of FIG. 1, disposed with the constraining collar (370). The collar (370) is not engaged with the jointed end (32) in FIGS. 11A and 11B and the jaws (310, 320) are in the open configuration. Retraction of the joined end into the collar (370) as shown in FIGS. 11A′ and 11B′ engages the constraining function of the constraining collar (370), thereby closing the jaws (310, 320). In FIGS. 11A and A′ the staple cartridge jaw (320) moves to close the jaws, whereas in FIGS. 11B and B′ anvil jaw (310) moves to close the jaws.

FIGS. 12A, A′ and 12B, B′ each depict a stapler head (302) as described herein containing the same or similar features as the stapler head (302) of FIG. 1, disposed with the closing screw (380). The closing screw (380) is not engaged with the jointed end (32) in FIGS. 12A and 12B and the jaws (310, 320) are in the open configuration. Advancement of closing screw (380) towards the opening end (34) shown in FIGS. 12A′ and 11B′ engages the closing function of the closing screw (380), thereby closing the jaws (310, 320). In FIGS. 12A and 12A′ the staple cartridge jaw (320) moves to close the jaws, whereas in FIGS. 12B and 12B′ anvil jaw (310) moves to close the jaws.

FIG. 13 shows the BDP (140) in a bent position and comprising a plurality of tandemly arranged articulated joints parts (400,a-f), each forming an articulated joint (400′,a-e). Each joint further constrains a radial position of a longitudinal member, LM (450,a-c). A maximum angle of bending (α_{d-e}) between two adjacent joints 400′,d and 400′,e is indicated. The maximum angle between two adjacent joint determines extent of bending of the drive shaft (460) passing through a lumen of each articulated joint part (400,a-f).

FIG. 14 is a side view of a part of an articulated joint part (400) that is an LM guide (402) in particular that is an articulated LM guide (405) having a disc shaped, and a distal side (40) and a proximal side (20). The articulated LM guide (405) has a body (402) comprising at the distal side (40, one component of the pair of components that forms a pivot joint that is a dome protrusion (430), akin to the ball of a ball and socket joint. It further comprises at the proximal side (20), the other component of the pair of components that forms a pivot joint that is a reciprocating recess (440), akin to the socket of a ball and socket joint. Further indicated is a pair of rotation limiters (432, 432′) fixedly connected to the dome protrusion (430), which are radial protrusions from said dome protrusion (430). These couple with a pair reciprocating slots (440, 440′) (rotationally and moveably) fixed in connection with the receiving recess (440) of an adjacent articulated LM guide (not shown), to prevent mutual axial rotation of adjacent articulated LM guides. Each articulated LM guide contains a discrete constraining point (i.e. a channel) and the pair of rotation limiters provide an essentially fixed mutual rotational alignment of the discrete constraining point along the fictive tube.

FIG. 15 is a plan view of a part of an articulated joint part (400) that is an LM guide (402) that is disc shaped. The LM guide (402) has a body (404) is disposed with 4 separate channels (410), arranged around a fictive tube (422). Each channel (410) constrains a LM (450). Each channel is regarded as a discrete constraining point. The body (404) of the LM guide (402) is also provided with a central lumen (420) in which the drive shaft (460) controlling the stapler head is disposed.

FIG. 16 is a plan view of a part of an articulated joint part (400) that is an LM guide (402) containing the same or similar features as the LM guide (402) of FIG. 15. In FIG. 15 the LMs (450,a-d) have a rectangular profile and complementary-profiled channels (410,1-d) and FIG. 16 the LMs (450,a-d) have a circular profile and complementary-profiled channels (410,1-d).

FIG. 17 is a cross-sectional view of a plurality of articulated joints parts (400a,b) each part being an LM guide (402a,b) containing the same or similar features as the articulated LM guide (405) of FIG. 14. Shown is a continuous lumen (428) formed from the plurality of individual lumens (422a,b) of each articulated LM guide (405), and the drive shaft (460) therein. Also shown are the dome protrusions (430a,b), akin to the ball of a ball and socket joint, and the other component of the pair of components that forms a pivot joint that is a reciprocating recess (440a,b) akin to the socket of a ball and socket joint.

FIG. 17A is an enlarged view of the continuous lumen (428) wherein each individual lumen (422a, 422b) is has a cylindrical walls flared (424, 426) at both ends.

FIGS. 18A and B show that each the lumen of each articulated joint part (400) (articulated LM guide (405)) is flared (424, 426) at both proximal and distal ends (FIG. 18A), and the bending (FIG. 18B) places the flexible part of the drive shaft (460) in the centre of the lumen (420) and avoids biting or locking its movements.

FIGS. 19A and B shows each the lumen of each articulated joint part (400) (articulated LM guide (405)) is uniformly cylindrical (FIG. 19A). Bending (FIG. 19B) causes the wall of the lumens to bite or lock (429) against the drive shaft (460) thereby restricting its movements.

FIG. 20 is a cross sectional view of a steerable fusing device (50) that is a steerable stapler (52), comprising a fusing head (300) that is a stapler head (302). The stapler head (302) is disposed with a cartridge jaw (320), anvil jaw (310), a threaded support member (322), a sled member (324), and a constraining member (350) that also incorporates a cutting blade. A drive shaft (460) that is a flexible cable is indicated.

FIG. 21 is a cross sectional view of a steerable fusing device (50) as shown in FIG. 20, except the drive shaft (460) is a segmented drive shaft (462 in the DBP (140).

FIG. 22 is a detail of FIG. 21 showing separate cylindrical articulated segments (461a-e) of the drive shaft each supported within an articulated joints part (400a-e).

FIG. 23 is a detail of a gear-type universal joint (464) between two articulated segments (461f-g) of the drive shaft.

FIG. 24 is a cross sectional of a segmented drive shaft showing separate cylindrical articulated segments (461h-m) that are spherical, the spheres each supported within an articulated joints part (400h-n) and also forming a pivoting joint.

FIG. 25 is an isometric view of a steerable fusing device that is a steerable stapler dismountably attached at the proximal end to a handle.

FIG. 26 is an isometric view of a steerable fusing device that is a steerable stapler dismountably attached at the proximal end to a fitting in connection with the effector end of a robotic arm.

FIG. 27 is an isometric view of a stapler head.

FIG. 28 is an isometric view of exploded parts of a stapler head.

1. A steerable fusing device (50) comprising:
a steerable instrument (100) having a proximal end (20) and a distal (40) end comprising a shaft (130), a bendable proximal part (120) configured to bend omnidirectionally in a curve and a bendable distal part (140) configured to bend omnidirectionally in a curve, the steerable instrument (100) configured such that the bendable distal part (140) bends responsive to bending of the bendable proximal part (120), and
a fusing head (302) attached in fixed rotational relation to the bendable distal part (140) configured for fusing tissue captured between jaws (310, 320) of the fusing head (300),
wherein the fusing head (300) is axially rotatable when the bendable distal part (140) is in a bent position by a complementary axial rotation of the bendable proximal part (120).

2. The steerable fusing device (50) according to claim 1, wherein steerable instrument (100) is further configured such that the direction of the fusing head (300) is changeable while the shaft is in a fixed rotational position by a complementary movement of the connector (110).

3. The steerable fusing device (50) according to claim 1, further comprising a connector (110) configured for dismountable attachment to a robotic arm, attached in fixed rotational relation to the bendable proximal part (120), wherein
the bendable distal part (140) bends responsive to bending of the bendable proximal part (120), and the stapler head (300) is axially rotatable when the bendable distal part (140) is in a bent position by a complementary axial rotation of the connector (110),
the shaft (130) is pivotable around a fulcrum zone (134) on the shaft (130) and changes direction responsive to a complementary movement of the connector (110),
thereby providing control of the shaft (130) direction, bending of the bendable distal part (140), and rotation of the end effector (150) through robotic movement of the connector (110).

4. The steerable fusing device (50) according to claim 3, wherein connector (110) comprises a rigid member for dismountable non-rotational attachment to a complementary fitting on the robotic arm, wherein the complementary fitting is disposed in fixed relation to a last joint of the robotic arm.

5. The steerable fusing device (50) according to claim 1, further provided with a drive shaft (460) attached at its distal end (40) to the stapler head (50) for transmission of force to control jaws (310, 320) of the stapler head (50).

6. The steerable fusing device (50) according to claim 5, wherein the BDP (130)
comprises a plurality tandemly arranged joints (400′, a-f), wherein the joints (400′, a-f) form an essentially continuous lumen (428) for passage of the drive shaft (460).

7. The steerable fusing device (50) according to claim 6 wherein each joint (400′, a-f) is formed from two articulating joint parts (400, a-f), each articulating joint parts (400, a-f) is provided with a separate lumen (422a, 422b) for passage of the drive shaft (460) wherein the proximal end (20), distal end (40) or both ends of the lumen (422a, 422b) wall flares outwards.

8. The steerable fusing device (50) according to claim 6, wherein a maximum bending angle of a joint (400′, a-f) is limited to 30°.

9. The steerable fusing device (50) according to claim 6, wherein a transverse profile of the drive shaft (460) is 25% to 99.8% of a transverse profile of the lumen (420) of the joint.

10. The steerable fusing device (50) according to claim 1, wherein the fusing head (300) is a stapler head (302) comprising a staple cartridge jaw (320) configured to support a staple cartridge holding a plurality of surgical staples (334) and an anvil jaw (310) disposed with an anvil plate, the anvil jaw (310) or staple cartridge jaw (320) being moveable with respect to the other jaw between an open and closed position.

11. The steerable fusing device (50) according to claim 9, wherein the staple cartridge jaw (320) or staple cartridge comprises a rotatable threaded support (322) and a sled member (324) whereby rotation of the rotatable threaded support (322) advances the sled member (324) to deploy staples (334) from the staple cartridge (320) and into the anvil plate.

12. The steerable fusing device (50) according to claim 9, further comprising a slidable constraining member (350) configured to move an open end (34) of the anvil jaw (310) or staple cartridge jaw (320) closer to the other jaw.

13. The steerable fusing device (50) according to claim 11, wherein slidable constraining member (350) is disposed in co-operation with the rotatable threaded support (322) such that rotation of the rotatable threaded support (322) advances the slidable constraining member (350) to move an open end (34) of the anvil jaw (310) or staple cartridge jaw (320) closer to the other jaw.

14. The steerable fusing device (50) according to claim 11, wherein the slidable constraining member (350) comprises a spacing beam (352) flanked by a pair of stop members (354, 356), where the spacing beam (252) is disposed in a slot (316, 326) of a body (318, 328) of each of the anvil jaw (310) and staple cartridge jaw (320) and each stop member (354,356) abuts the slot edges (316a,b, 326a,b) thereby retaining the anvil jaw (310) and staple cartridge jaw (320) together at a distance determined by spacing beam (352).

15. The steerable fusing device (50) according to claim 1, wherein the staple cartridge jaw (320) and anvil jaw (310) are connected at a jointed end (32) to one or more joints (340) allowing movement of the jaws (310, 320) between an open and closed position, wherein the jointed end (32) is disposed proximal (20) or distal (40) to an opening end (34) of the jaws.

16. A steerable fusing device (50) comprising:
a steerable instrument (100) having a proximal end (20) and a distal (40) end comprising a shaft (130), a bendable distal part (140) configured to bend omnidirectionally in a curve, a set of longitudinal members, LMs, configured to transmit actuating movement along the shaft (130) to the bendable distal part (140), the steerable instrument (100) configured such that the bendable distal part (140) bends responsive to actuation of the proximal part (120), and
a fusing head (302) attached in fixed rotational relation to the bendable distal part (140) configured for fusing tissue captured between jaws (310, 320) of the fusing head (300).

17. The steerable fusing device (50) according to claim 16, wherein a proximal end of the set of LMs is configured for detachable coupling to a set of actuators, wherein each actuator in the set of actuators controls movement of one or more LMs, thereby controlling bending of the bendable distal part (140).

18. The steerable fusing device (50) according to claim 17, wherein at least one actuator in the set of actuators is a servo motor, linear actuator, hydraulic actuator, or pneumatic actuator.

19. The steerable fusing device (50) according to claim 17, wherein the set of actuators is incorporated into a complementary fitting of a robotic arm.

20. The steerable fusing device (50) according to claim 17, further provided with a drive shaft attached at its distal end to a stapler head for transmission of force to control jaws of the stapler head.

21. A system comprising a robotic arm and a steerable fusing device (50) according to claim 1.
